(19) United States
(12) Patent Application Publication
(10) Pub. No.: US 2025/0000526 A1
(43) Pub. Date: Jan. 2, 2025

(54) METHOD OF AND APPARATUS FOR FIXING AN IMPLANT WITH RESPECT TO FIRST AND SECOND BONE PARTS

(71) Applicant: TriMed Inc., Santa Clarita, CA (US)

(72) Inventor: Robert J. Medoff, Kailua, HI (US)

(21) Appl. No.: 18/759,310

(22) Filed: Jun. 28, 2024

Related U.S. Application Data
(60) Provisional application No. 63/524,146, filed on Jun. 29, 2023.

(51) Int. Cl. A61B 17/17; A61B 17/00; A61B 17/16; A61F 2/46

(52) U.S. Cl. CPC A61B 17/17; A61B 17/1622; A61F 2/4603; A61B 2017/00982

(57) ABSTRACT

A method of, and apparatus for, fixing an implant with respect to first and second bone parts at a joint. The method includes the steps of: obtaining a guide structure having a body with a first discrete projecting referencing arm; placing the guide structure in an operative position by directing the first discrete projecting referencing arm through a joint capsule to against a joint surface on the first bone part to establish a referencing location relative to the joint surface; with the guide structure in the operative position, using the guide structure to facilitate alignment of the implant in a desired seated position with respect to the first bone part; and thereafter fixing the implant in the desired seated position.

### CROSS-REFERENCE TO RELATED APPLICATIONS

This is a non-provisional application claiming priority to U.S. Provisional Application No. 63/524,146, filed Jun. 29, 2023, which is incorporated herein by reference.

### BACKGROUND OF THE INVENTION

### Field of the Invention

This invention relates to bone implants and, more particularly, to a method of, and apparatus for, fixing an implant to a bone to thereby fix a first bone part/fragment relative to a second bone part at a joint location.

### Background Art

Many implants have been designed to provide fixation near a joint location using fixation elements, such as screws, pegs, legs, hooks, etc., that penetrate the bone next to the joint and are oriented at a specific angle to provide optimal placement. These fixation elements, near the joint surface in many of these implants, are often locked at a preferred fixed position relative to the rest of the implant, which is commonly contoured to match the complex curve of the bone surface. The entry location and angular orientation of the fixing elements must be selected so that when the implant is applied to the surface of the bone for which it is designed, these fixation elements near the joint surface will be optimally directed and positioned relative to the joint surface. In the event that an articular fragment is shifted to an angular malposition, the surgeon will intentionally modify the angle of the formed holes/bores for the fixation elements so as to correct for the angular malposition of the fragment when the implant is applied. Guessing the specific entry locations for the bores relative to the joint surface as well as the angular orientation of the trajectory of the bores that matches the proximal/distal and medial/lateral orientation can be problematic for the surgeon.

In the zone between the metaphyseal bone surface and the actual joint surface, there is often a rim or edge that defines a region where the surface bone transitions to the joint surface. Typically, this rim or edge receives the attachments of the joint capsule and ligament structures, making visualization and identification of the joint surface difficult. Generally, the surgeon does not want to compromise these soft tissue structures as they are important to the stability of the joint. Although an implant may be designed for placement of both the implant and fixation elements at designated positions relative to the joint, the surgeon can often only visualize a limited bone surface area and resultingly may have a limited frame of reference when attempting to form bone bores for the implant and fixation elements that will cause the implant ultimately to be optimally positioned with respect to the bone for which it is designed. Consequently, the entry locations for bores for the fixation elements may be selected by the surgeon based simply on the combination of initial best judgment and subsequent trial and error. More specifically, a guess is made for the starting position and trajectory of drilled bores for fixation elements and then checked with x-ray. The entry location and/or trajectory may then be modified repeatedly until an optimal final entry location and trajectory are accepted.

This process may take a considerable amount of time, which may lead to surgeon and surgical team fatigue as well as cause increased exposure of the wound to bacterial contamination and possibly infection. Further, repetitive penetration of the bone during the trial-and-error process may compromise the bone at the operating site.

Further, if separate drilled bone bores are required for multiple fixation elements arranged side-by-side, which is common, the relative proximal/distal position of each bore starting point needs to be precisely established in order to align the long axis of the implant with the longitudinal axis of the bone shaft. If not correctly positioned, the implant will be obliquely oriented to the longitudinal axis of the bone, causing the contour of the implant to be incongruent with the contour of the applied bone surface. This may create further challenges in achieving bone fixation for the surgeon.

The above problems persist, particularly at all joint locations where there is a large, curved bone rim configuration that is covered by joint membrane and ligament attachments, which make it difficult to precisely identify and set the optimal entry location and trajectory of fixation elements. In the event that the implant is not properly positioned with respect to bone parts, a procedure may be compromised.

### SUMMARY OF THE INVENTION

In one form, the invention is directed to a method of fixing an implant with respect to first and second bone parts at a joint with an adjacent bone. The first and second bone parts make up one bone with a length. The implant has a bone engaging surface. The method includes the steps of: obtaining a guide structure having a body with at least a first discrete projecting referencing arm; placing the guide structure in an operative position by causing the at least first discrete projecting referencing arm to penetrate a joint capsule between the first bone part and the adjacent bone and position adjacent a joint surface on the first bone part to abut the joint surface on the first bone part so as to thereby limit shifting of the guide structure relative to the first bone part lengthwise of the one bone and provide a referencing location with respect to the joint surface on the first bone part; with the guide structure in the operative position, using the guide structure to facilitate alignment of the implant in a desired seated position wherein the bone engaging surface on the implant is in confronting relationship with a surface on the one bone; and fixing the implant in the desired seated position.

In one form, the step of using the guide structure to facilitate alignment of the implant in a desired seated position involves abutting the implant to the guide structure with the guide structure in the operative position.

In one form, the step of using the guide structure to facilitate alignment of the implant in a desired seated position involves using the guide structure to guidingly direct a drilling tool into the first bone part with the guide structure in the operative position.

In one form, the step of using the guide structure to facilitate alignment of the implant in a desired seated position further includes the step of abutting the implant to the drilling tool with the drilling tool directed into the first bone part.

In one form, the step of using the guide structure to facilitate alignment of the implant in a desired seated position involves separating the drilling tool from the first bone part so that a bore formed by the drilling tool in the first bone part is exposed, and using the bore to one of: a) accept a fastener that can be directed through the implant to fix the implant in the desired seated position; and b) accept a projecting part on the implant.

In one form, with the guide structure in the operative position a surface portion on the guide structure bears against a selected region of a surface on the first bone part at a location spaced from a location at which the at least first discrete projecting reference arm abuts the joint surface on the first bone part.

In one form, the guide structure has a contoured surface configured to bear against a selected region of a surface on the first bone part. The contoured surface is defined at least in part by the at least first discrete projecting referencing arm and at least nominally matched in shape to the selected region on the surface of the first bone part.

In one form, with the guide structure in the operative position the contoured surface is placed in confronting relationship with the selected region on the surface of the first bone part.

In one form, the guide structure has at least a first guide passage. The step of using the guide structure to facilitate alignment of the implant in the desired seated position involves: a) directing a drilling tool through the first guide passage and into the first bone part with the guide structure in the operative position; and b) with the drilling tool directed into the first bone part extending the drilling tool into an opening in the implant whereby the drilling tool abuts the implant to align the implant along the length of the one bone.

In one form, the guide passage has an axis. With the guide structure in the operative position, the guide passage axis intersects the first bone part at a predetermined angle with respect to the first bone part. The step of directing the drilling tool through the first guide passage involves directing the drilling tool guidingly through the first guide passage along the guide passage axis.

In one form, the method further includes the step of maintaining the guide structure in the operative position before using the guide structure to facilitate alignment of the implant in the desired seated position.

In one form, the step of maintaining the guide structure in the operative position involves directing a drilling tool through a second guide passage in the guide structure and into the first bone part.

In one form, the implant has at least one projecting part. The guide structure body has at least a first guide passage with an axis. With the guide structure in the operative position, the axis of the first guide passage intersects the first bone part at a predetermined location spaced from the referencing location lengthwise of the one bone and at a predetermined angle with respect to the first bone part. The step of using the guide structure to facilitate alignment of the implant involves: a) maintaining the guide structure in the operative position; and b) with the guide structure maintained in the operative position directing a drilling tool guidingly through the first guide passage to form a first bore in the first bone part. The method further includes the step of advancing the one projecting part on the implant into the first bore to place the implant in the desired seated position. The step of fixing the implant involves fixing the implant to the second bone part.

In one form, the method further includes the step of repositioning the implant to bring a part of the bone engaging surface on the implant into confronting relationship with a surface on the second bone part and thereby cause the first bone part to be repositioned relative to the second bone part.

In one form, the at least first discrete projecting referencing arm has a sharp free end.

In one form, the at least first discrete projecting referencing arm tapers towards a free end.

In one form, the guide structure has a second discrete projecting referencing arm spaced from the first discrete projecting referencing arm and configured to penetrate the joint capsule as the guide structure is placed in the operative position.

In one form, the guide structure has a contoured surface configured to bear against a selected region of a surface on the first bone part and the contoured surface has a hook-shaped portion to extend over the selected surface region on the first bone part.

In one form, the guide structure body has a block portion through which the first guide passage is formed. There is a second guide passage formed through the block portion in spaced relationship with the first guide passage. The method further includes the step of directing a drilling tool through the second guide passage and into the first bone part to define a second bore in the first bone part.

In one form, there is a third guide passage formed through the block portion in spaced relationship to the first and second guide passages. The method further includes the step of directing a drilling tool through the third guide passage and into the first bone part.

In one form, the guide structure further includes an elongate handle projecting in cantilever fashion. The elongate handle has a portion configured to be grasped by a user to facilitate holding and repositioning of the guide structure body.

In one form, the portion configured to be grasped on the elongate handle has a gripping length projecting in a line that is angled with respect to the axis of the first guide passage.

In one form, the guide structure body has a guide portion through which the first guide passage is formed. The elongate handle has an “L” shape with first and second legs. The first leg is connected to the block portion and the second leg extends from the first leg and defines the gripping length.

In one form, the step of placing the guide structure in the operative position involves determining an optimal orientation of a line of the first bore relative to the first bone part and selecting the operative position of the guide structure so that the first guide passage axis is substantially coincident with the line of the first bore.

In one form, the step of placing the guide structure in the operative position involves repositioning the guide structure relative to the first bone part with the at least first discrete projection penetrating the joint capsule to thereby change an orientation of the line of the first bore relative to the first bone part to arrive at the optimal orientation of the line of the first bore relative to the first bone part.

In one form, the step of determining an optimal orientation of a line of the first bore involves making a determination while inspecting the first bone part with x-ray.

In one form, the step of advancing the one projecting part on the implant into the first bore involves guiding the one projecting part along the drilling tool through one of: a) direct interaction of the implant and drilling tool; and b) interaction of the implant and an attachment on the implant.

In one form, the drilling tool is separated from the first bone part before advancing the one projecting part on the implant into the first bore.

In one form, the step of repositioning the implant involves directly engaging the implant and exerting a force upon the implant to effect repositioning.

In one form, the method further includes the step of anchoring the guide structure before advancing the one projecting part on the implant into the first bore.

In one form, the step of anchoring the guide structure is performed by directing a drilling tool through one of the first and second guide passages and into the first bone part.

In one form, the guide structure is separated from the first bone part before advancing the one projecting part on the implant into the first bore to place the implant in the desired seated position.

In one form, the one projecting part on the implant is advanced into the first bore with the guide structure in the operative position.

In one form, the implant is in the form of one of: a) a plate with the at least one projecting part having a hook shape; b) a fixed angle plate; c) a plate with the at least one projecting part comprising a fixed projecting part; and d) a body comprising a formed wire.

In one form, the implant is guided by the drilling tool towards the desired seated position for the implant.

In one form, the guide structure body has a surface that extends only part way around the axis of the first guide passage so that with the drilling tool extending into the first guide passage the guide structure body can be separated from the drilling tool by effecting relative movement of the drilling tool and guide structure body in a direction transverse to the axis of the first guide passage.

In one form, the method further includes the step of anchoring the guide structure by directing a drilling tool into one of the guide passages.

In one form, the at least first discrete projecting referencing arm has a free end. The axis of the first guide passage is spaced a distance from the free end of the at least first discrete projecting referencing arm.

In one form, the guide structure is configured so that the distance between the axis of the first guide passage and free end of the at least first discrete projecting referencing arm can be selectively changed.

In one form, the one projecting part is directed through the first guide passage as the one projecting part is advanced into the first bone part.

In one form, the method further includes the step of providing a kit including at least one of: a) different forms of the implant; b) different forms of the guide structure; c) different forms of the drilling tool; and d) additional components usable to perform the method described above.

In one form, the first bone portion defines a rim. The hook-shaped portion extends over the surface on the first bone part at the rim.

In one form, the gripping length is oriented so that with the gripping length aligned with the length of the second bone part, the first bone is located and aligned so that with the implant thereafter fixed in the seated position, there is coaptation of the bone engaging surface on the implant along a surface of the second bone part.

In one form, the gripping length is oriented so that with the gripping length aligned with a length of the second bone part and the implant thereafter fixed in the desired seated position, a length of the implant aligns with a length of the second bone part.

In one form, the guide structure in the operative position is configured to identify a position of an end of the implant with the implant placed in the desired seated position performing the steps described above using the guide structure in the operative position.

In one form, there is a discrete mark on the guide structure facilitating positioning of the end of the implant with the implant placed in the desired seated position.

In one form, the guide structure in the operative position is configured to identify an orientation of a length of the implant with the implant placed in the desired seated position performing the steps described above using the guide structure in the operative position.

In one form, the guide structure has an elongate bar that with the guide structure in the operative position projects in a direction substantially aligned with a length of the implant with the implant placed in the desired seated position performing the steps described above using the guide structure in the operative position.

In one form, the method further includes the steps of: obtaining an adaptor having a body with another guide passage with an axis; and connecting the adaptor to the guide structure so that the adaptor is in an assembled position wherein the axis of the another of the guide passages intersects the first bone part at a location spaced from a location at which the axis of the first guide passage intersects the first bone part.

In one form, there are connectors on the adaptor and the guide structure that cooperate to consistently maintain a preselected relationship between the adaptor and guide structure with the adaptor in the assembled position.

In one form, the method further includes the steps of obtaining an adaptor having a body with an alignment component and another guide passage with an axis, connecting the adaptor to the guide structure body by directing the alignment component into one of the first and second guide passages, and moving the adaptor so that the alignment component advances guidingly in the one of the first and second guide passages until the adaptor achieves an assembled position wherein with the guide structure in the operative position and the adaptor in the assembled position the axis of the another of the guide passages intersects the first bone part at a location spaced from each of: a) a location that the axis of the first guide passage intersects the first bone part, and b) a location that an axis of the second guide passage intersects the first bone part.

In one form, the location at which the axis of the another of the guide passages intersects the first bone part is spaced further away from the referencing location than the location at which at least one of the axes of the first and second guide passages intersects the first bone part.

In one form, the location at which the axis of the another guide passage intersects the first bone part is spaced further away from the referencing location than the location at which each of the axes of the first and second guide passages intersects the first bone part.

In one form, the implant guide structure body has a second guide passage. The method further includes the steps of: directing a drilling tool guidingly through the second guide passage to form a second bore in the first bone part; removing the drilling tool from the first bore; with the drilling tool remaining in the second bore, directing the drilling tool remaining in the second bore into the one implant opening and thereafter guiding the implant along the drilling tool remaining in the second bore to thereby advance the one projecting part up to the first bore and thereafter into the first bore to place the bone engaging surface on the implant in confronting relationship with the surface on the first bone.

In one form, the method further includes the step of connecting the body of the guide structure to the implant.

In one form, the method further includes the step of placing the implant against the one bone after the body of the guide structure is connected to the implant.

In one form, the step of connecting the body of the guide structure to the implant involves the steps of connecting a block guide to the implant and connecting the body of the guide structure to the block guide.

In one form, the method further includes the step of separating the body of the guide structure from the implant with the implant in the desired seated position.

In one form, the step of connecting the body of the guide structure to the implant is performed using at least one separate fastener.

In one form, the at least one separate fastener includes one separate fastener that connects the block guide to the implant.

In one form, the at least one separate fastener includes another one of the at least one of the separate fasteners that connects the body of the guide structure to the block guide.

In one form, the method further includes the step of separating the block guide from the implant with the implant in the desired seated position.

In one form, the at least one separate fastener is a threaded fastener.

In one form, the step of connecting the body of the guide structure to the block guide involves press connecting the body of the guide structure to the block guide.

In one form, the step of press connecting the body of the guide structure to the block guide involves engaging snap fit connectors on the body of the guide structure and the block guide.

In one form, the guide structure is provided in combination with a drilling tool configured to be guided by the first guide passage and in the form of one of: a) a rotary drill bit; and b) a pin.

In one form, the guide structure body has a second guide passage with an axis. With the guide structure in the operative position, the axis of the second guide passage intersects the first bone part at a location on the first bone part spaced further away from the referencing location lengthwise of the bone than a location at which the axis of the first guide passage intersects the first bone part.

In one form, the invention is directed to the guide structure as used in performing the methods described above.

### BRIEF DESCRIPTION OF THE DRAWINGS

FIG. 1 is a schematic representation of a joint location with a fractured bone with a first articular bone part/fragment correctly anatomically positioned relative to a second/stable bone part from which it was separated;

FIG. 2 is a view as in FIG. 1 wherein the first bone part is malpositioned relative to the second bone part;

FIG. 3 is a view as in FIG. 2 and showing alternative locations at which a surgeon might place a distal region of an implant using conventional techniques;

FIG. 4 is a perspective view of one form of guide structure, according to the invention, usable to perform a method of fixing an implant with respect to first and second bone parts;

FIG. 5 is a view of bone parts as in FIG. 2 with the inventive guide structure in FIG. 4 being moved towards an operative position with respect thereto;

FIG. 6 is a view as in FIG. 5 with the guide structure optimally oriented in an operative position;

FIG. 7 is a view as in FIG. 6 wherein a drilling tool has been directed through a guide passage on the guide structure to anchor the guide structure in the operative position;

FIG. 8 is a view as in FIG. 7 wherein a drilling tool is guided by the guide structure to produce additional bores in the first bone part;

FIG. 9 is a view as in FIG. 8 wherein projecting parts/legs on an implant have been directed through guide passages on the guide structure and into the bores formed in the first bone part;

FIG. 10 is a view as in FIG. 9 wherein the guide structure has been separated from the bone parts and the implant has been fully seated with respect to the first bone part;

FIG. 11 is a view as in FIG. 10 wherein the implant has been manipulated to bear a proximal region of the implant against the second bone part, thereby anatomically correcting the relationship between the first bone part and second bone part, and with the implant fixed to the second bone part through fasteners;

FIG. 12 is a view as in FIG. 9 using another form of implant wherein the guide structure has been separated preparatory to advancing of the implant towards a seated position;

FIGS. 13 and 14 correspond to FIGS. 10 and 11, with FIG. 13 showing the implant in FIG. 12 fully seated with respect to the first bone part and FIG. 14 showing the implant against and fastened to the second bone part;

FIG. 15 is an exploded, perspective view of the guide structure in FIG. 4;

FIG. 16 corresponds to FIG. 17 and shows a different form of guide structure;

FIG. 17 is a view as in FIG. 16 wherein another form of implant has been fixed to the first and second bone parts with the guide structure in FIG. 16 removed;

FIG. 18 is a flow diagram representation of a method of fixing an implant with respect to first and second bone parts, according to the invention;

FIG. 19 is a schematic representation of the inventive guide structure, with the guide structure in FIGS. 4-17 being a representative form;

FIG. 20 is a schematic representation of a kit including different combinations of components that can be kept at an operating site to practice the inventive method while adapting to different conditions at the operating site;

FIG. 21 is a schematic representation of a modified form of guide structure incorporating an adjusting capability and indicia to facilitate proper placement of an implant;

FIG. 22 is a schematic representation of a further modified form of guide structure with guide passages spaced from each other lengthwise of a bone in which the guide structure is in an operative position;

FIG. 23 is a partially fragmentary, perspective view of a modified form of guide structure, according to the present invention, with an adaptor being connected thereto to define guide passages spaced from the guide passages on the guide structure;

FIG. 24 is a partially schematic, end elevation view of the adaptor in FIG. 23;

FIG. 25 is a partially schematic, plan view of the adaptor in FIGS. 23 and 24;

FIG. 26 is an exploded, perspective view of the guide structure and adaptor in FIGS. 23-25;

FIG. 27 is an exploded, perspective view of a modified form of guide structure and adaptor, according to the present invention;

FIG. 28 is a view as in FIG. 27 with the guide structure components assembled and the adaptor in an assembled position relative to the guide structure;

FIG. 29 is a view as in FIG. 28 from a different perspective;

FIG. 30 is a view as in FIGS. 28 and 29 from a still further different perspective;

FIG. 31 is an enlarged, plan view of the components in the state in FIGS. 28-30;

FIG. 32 is an enlarged, plan view of the guide structure in FIGS. 27-31 with the adaptor removed;

FIG. 33 is a perspective view of the guide structure in FIG. 32 from a different perspective;

FIG. 34 is a schematic representation of a further modified form of the inventive guide structure usable in conjunction with another form of implant;

FIG. 35 is a fragmentary, elevation view showing another form of guide structure, according to the invention, in an operative position with respect to a fractured bone;

FIG. 36 is a view as in FIG. 35 wherein an implant has been directed into a desired seated position by being abutted to a part of the guide structure;

FIG. 37 is a fragmentary view of the guide structure in the operative position of FIG. 35 and from a different perspective;

FIG. 38 is a side elevation view of a further modified form of guide structure being advanced towards an operative position relative to a fractured bone;

FIG. 39 is a view as in FIG. 38 with the guide structure in the operative position;

FIG. 40 shows the guide structure in the operative position in FIG. 39 and from a different perspective;

FIG. 41 is a fragmentary view of the operatively positioned guide structure as in FIG. 40 and with a drilling tool advanced through the guide structure and into the bone;

FIGS. 42-45 sequentially show the structure in FIG. 39 being used to place one form of implant in a desired seated position and to effect fixation of the implant in the desired seated position;

FIGS. 46-51 sequentially show the structure in FIG. 39 being used to place a different form of implant in a desired seated position and to effect fixation of the implant in the desired seated position;

FIGS. 52-57 sequentially show the structure in FIG. 39 being used to place still another form of implant in a desired seated position and to effect fixation of the implant in the desired seated position;

FIG. 58 is an elevation view of a still further modified form of guide structure, according to the invention;

FIG. 59 is a further flow diagram representation of a method of fixing an implant with respect to first and second bone parts, according to the invention;

FIG. 60 is a schematic depiction of another form of guide structure that is releasably connectable to an implant;

FIG. 61 is an exploded perspective view of a conventional block guide utilized in conjunction with an implant;

FIG. 62 is a plan view of an exemplary form of the guide structure as in FIG. 60 operatively positioned on, and releasably connected to, the implant in FIG. 61 with the block guide thereon;

FIG. 63 is a side elevation view of the components in FIG. 62;

FIG. 64 is an end elevation view of the components in FIGS. 62 and 63;

FIG. 65 is a plan view of the implant in FIGS. 62-64

FIG. 66 is a plan view of the block guide in FIGS. 62-64;

FIG. 67 is a plan view of the guide structure in FIGS. 62-64;

FIG. 68 is a plan view of a coupling bolt usable to releasably maintain the guide structure in an operative position;

FIG. 69 is an exploded, side elevation view of the components in FIGS. 62-64;

FIG. 70 is an end perspective view of the block guide in FIGS. 62-64, FIG. 66, and FIG. 69;

FIG. 71 is an end elevation view of the block guide in FIGS. 62-64, FIG. 66, and FIG. 69;

FIG. 72 is an end elevation view of the guide structure in FIGS. 62-64, FIG. 67, and FIG. 69;

FIG. 73 is a side elevation view of the coupling bolt in FIGS. 62-64, FIG. 68, and FIG. 69;

FIG. 74 is a perspective view of a modified form of guide structure, according to the invention;

FIG. 75 is a perspective view of a further form of guide structure, according to the invention; and

FIG. 76 is an exploded perspective view of the guide structure in FIG. 75.

### DETAILED DESCRIPTION OF THE PREFERRED EMBODIMENT

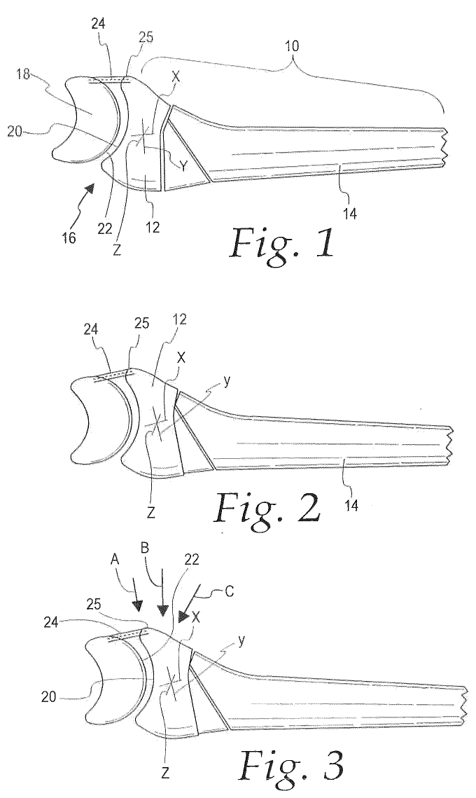

In FIGS. 1-3, a fracture site adjacent a joint is depicted generically with it demonstrated with respect thereto the challenge of selecting the optimal bone entry location and angular orientation of a fixation element for an implant.

In FIG. 1, a fractured bone 10 is depicted, with the fracture producing at least a first, articular bone part 12 and a second bone part 14. The first bone part 12 is located at a joint 16 defined at least in part by the first bone part 12 and a second, adjacent, bone 18. Joint surfaces 20, 22, respectively on the first bone part 12 and second bone 18, are covered in part with cartilage and are guided in movement against and relative to each other. For purposes of simplicity, the cartilage will be considered to be part of the bones at the joint surfaces 20, 22. A joint capsule 24 overlies the joint 16 and is shown at one side thereof. The first bone part 12 has a rim/edge 25 adjacent the joint surface 20 to which the joint capsule 24 and ligament structures (not shown) attach.

In FIG. 1, the first bone part 12 and second bone part 14 are shown in their optimal relationship. An orientation reference system for the bone 10 is shown with x, y, and z axes.

As depicted, the x axis is substantially parallel to the length of the bone 10 and the second bone part 14. Various implants accommodating the contours of the bone 10 are designed using such a frame of reference.

The nature of the fracture may be such that, as described in the Background portion above, the surgeon is observing the FIG. 2 site condition with limited bone surface exposure and with the first/articular bone part 12 turned into an angular malposition in relationship to the second bone part 14. This angular malposition may be around a single axis or multiple axes. As depicted, the first bone part 12 is turned from its optimal position in FIG. 1 principally around the z axis whereby the y axis, which is optimally substantially orthogonal to the length of the second bone part 14, is at a different angle.

As depicted in FIG. 3, not only is the surgeon handicapped by not being able to determine the particular, degree of, or angle of, malposition, he/she is also challenged to determine precisely where the distal end of an implant should be placed with respect to the first bone part 12 to match contours. As shown in FIG. 3, three exemplary possible locations of the distal end of the implant are identified through the arrows A, B, C.

Improper placement of the distal end of the implant and/or improper orientation may result in suboptimal support of the adjacent joint surface with loss of reduction and deformity as well as mismatching of contours of implant surfaces and cooperating bone surfaces which the implants are specifically designed to engage in a precise manner.

In short, the surgeon is commonly left with no other options than to use a reasonable judgment as to the entry location and angular orientation of a fixation element for the particular implant based upon the observed site condition and the perceived malpositioning of the first bone part 12. As noted above, this may be a process that involves making a judgment, directing a fixing element into the first bone part 12, and evaluating the selected entry location and trajectory, as by radiographic imaging/x-ray. Failure to select the optimal entry location and trajectory may necessitate a removal and reinsertion of the particular fixation element.

Such trial and error processes can result in malposition of the implant, increased operating time, surgeon fatigue, increased risk of infection, and eventual compromised fixation of the bone and/or tissue at the operating site.

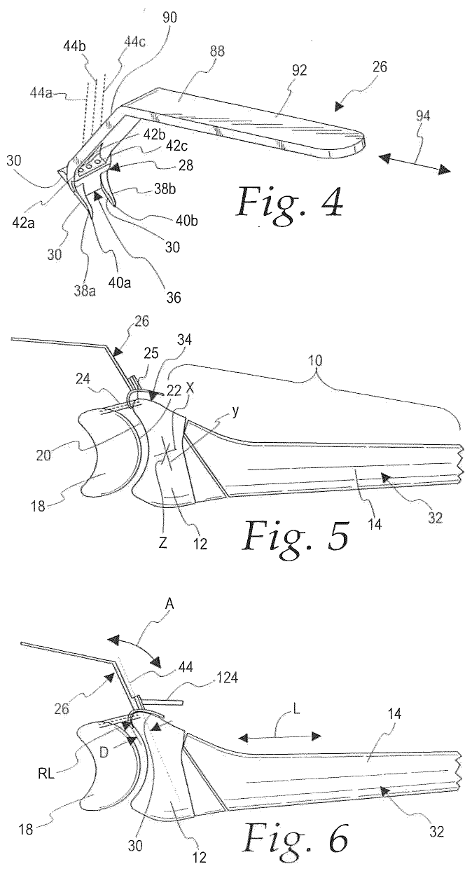

In FIGS. 4-15, an exemplary form of guide structure 26, according to the invention, is shown used in performing a method of fixing an implant with respect to corresponding first and second bone parts 12, 14, of the nature described with respect to FIGS. 1-3. The guide structure facilitates alignment of the implant in a desired position with respect to a fractured bone for which the implant is designed.

This form of the guide structure 26, and other forms of the guide structure, described below, facilitate alignment of different forms of implants by being used in multiple different ways.

The bone parts 12, 14 making up the bone 10, and bone 18, are shown with a generic shape that is not limited to any joint location, since the inventive concept is not limited to any particular implant shape or style or use at any joint location.

The guide structure 26 consists of a body 28 having a surface 30 configured to bear against a selected region of a surface on the first bone part 12. The surface 30 is contoured generally as a non-flat surface, but could be made to function with the surface 30 being flat, or having flat surface portions. As shown in FIGS. 5-14, the bone 10 has an overall exposed surface 32 including a portion defined by the first/articular bone part 12. As depicted, the selected surface region is identified at 34 and extends from the joint surface region 20 in a curved shape, as seen from the depicted perspective, up to and over the rim/edge 25. Typically, the surface at the rim/edge 25 will not be visible to the surgeon, nor will the joint surfaces 20, 22 be visible.

Generally, the selected surface region 34 has an overall inverted “U” or convex shape, with the bone 10 viewed as in the Figures and sectioned by a plane containing a lengthwise axis of the bone 10, with the contoured surface 30 on the guide structure body 28 at least nominally matched in shape to the surface region 34. At some sites, the selected surface region 34 may have a shape more like an edge joining two surfaces. While the rim/edge 25 and joint surfaces 20, 22 will generally not be visible during a procedure, for simplicity, they will be considered herein to make up part of the surface region 34.

The characterization of the contour matching as “nominal” is intended to encompass general contour matching. When the contoured surface 30 is borne against the surface region 34, there is at least a degree of contact between contoured surfaces on the guide structure body 28 and bone 10 which allows the body 28 to be generally maintained in a selected position relative to the first bone part 12—providing resistance to angular movement relative to the first bone part 12 as well as blocking translational movement of the guide structure body 28 along the bone 10 away from the joint location.

The matching shapes need not be continuously coextensive. For example, spaced point contact locations on the guide structure body 28 might cooperatively define a receptacle for a part of the first bone part 12, including at the rim/edge 25. This structure functions equivalently to continuously coextensive matching surface shapes and will thus be considered to provide nominally matched, contoured surfaces.

As explained below with respect to a modified form of the guide structure, cooperating contoured surfaces are not required. What is important is that the guide structure have at least one contact location with the first bone part 12 so as to define a referencing location RL (FIG. 6), as explained further below.

In the depicted form, the body 28 has a block portion at 36 and first and second discrete projecting referencing arms 38a, 38b, cantilevered away from the block portion 36. As depicted, the discrete projecting referencing arms 38a, 38b have the same shape, but this is not required.

As depicted, the discrete projecting referencing arms 38a, 38b have sharp free ends 40a, 40b, respectively. The free ends 40a, 40b are sharp to allow penetration of tissue without significant damage thereto and, as depicted, are in the form of sharp points. This is not a required shape.

As further depicted, the discrete projecting referencing arms 38a, 38b taper towards their respective free ends 40a, 40b. The projecting lengths of the discrete projecting referencing arms 38a, 38b have curved undersides which each defines part of the contoured surface 30.

With the discrete projecting referencing arms 38a, 38b in spaced relationship and configured as depicted, they cooperatively define, alone or in conjunction with at least part of the block portion 36, a hooked configuration which can be pressed, free ends first, against the joint capsule 24 to effect penetration thereof, whereupon the hook-shaped portion of the contoured surface 30 will receive part of the surface region 34 on the first bone part 12. The discrete projecting referencing arms 38a, 38b are situated adjacent the joint surface 20 whereby the surface 30 blocks movement of the guide structure 26 relative to the first bone part 12 in a direction along the length of the bone 10 indicated by the double-headed arrow L—left-to-right in FIGS. 5 and 6. “Adjacent”, as used herein, is intended to encompass relationships wherein the referencing arms 38a, 38b are in proximity to the joint surface 20—whether side-by-side, above, etc. This represents either a preliminary operative position or a final operative position for the guide structure, as shown in FIG. 6. As explained further below, with the portion of the surface 30 defined by the discrete projecting referencing arms 38a, 38b bearing against the first bone part 12, a referencing location RL is established relative to the joint surface 20, away from which distances lengthwise relative to the bone 10 can be gauged. The spaced referencing arms 38a, 38b also provide some stabilization of the guide structure 26 against skewing about an axis generally aligned with the length of the referencing arms 38a, 38b.

To stabilize the guide structure 26 against lengthwise shifting, the contact at the referencing location RL on the first bone part 12 may be made through point contact, as at the free ends of the referencing arms 38a, 38b, or over a larger area on the guide structure body 28. For purposes of simplicity throughout the description and claims herein, the contacting region on the guide structure body—whether a point or a larger area—will be characterized as a “surface.” This surface-to-surface contact provides the basic referencing location function, whereas cooperating contoured surfaces may afford additional stabilization and may facilitate guided adjusting movement of the guide structure 26 relative to the first bone part 12, to place the guide structure 26 in different operative positions, as described in greater detail below.

As also explained below, a single referencing arm might be utilized and may have a sharp free end or another free end configuration that contacts the first bone part 12.

The block portion 36 on the body 28, in the form depicted, has three spaced through guide passages 42a, 42b, 42c with substantially parallel axes 44a, 44b, 44c, successively.

The orientation of the axes 44a, 44b, 44c and location of the same relative to the body 28 and surface 30 are dictated by the specific bone at the site of application as well as the configuration of the implant to be used at the site.

For example, the guide structure 26 is configured so that with the body surface 30 placed against the surface region 34 on the first bone part 12, the guide passages 42 can be oriented so that the entry point to the first bone part 12 and orientation of the axes 44 can be optimized to facilitate the placement of fixation elements to optimally maintain the particular implant selected in a desired angular orientation and with the distal end thereof optimally situated relative to the joint surface 20 of the first bone part 12.

As can be seen in FIG. 6, the axes 44 are offset from the free ends 40 of the discrete projecting referencing arms 38 a distance D. As depicted, the axes 44 reside in a common plane. However, this is not required.

The angular position of the guide structure 26 can be adjusted, as indicated by the double-headed arrow A in FIG. 6, and assessed with radiographic imaging/x-ray, to change where the axes align at the surface of the bone 10 and the angular relationship of the axes 44 and bone part 12, to arrive at a final operative position for the guide structure 26, thereby to control the placement of fixation elements at precisely the correct location and orientation to facilitate implant placement and fixation, as described below. For example, the angle and locations of the axes 44 can be evaluated under radiographic guidance/x-ray once a drilling tool 46 is advanced through one of the passages 42 into the bone part 12.

Once the guide structure 26 is placed into the operative position with the projecting referencing arms establishing the joint referencing location RL on the joint surface and a spaced location on the surface 30 abutted to the rim/edge 25, the guide structure can be used to direct the positioning of an implant as well as assist in establishing the entry location and/or trajectories of fixation elements for the implant in relation to the joint surface of the bone part 12. The cooperating contoured surfaces on the guide structures 28 and first bone part 12 facilitate angular repositioning of the guide structure 26 and potentially a shifting of the referencing location RL by the surgeon, as required. Depending on the form of the implant used, the technique following placement of the guide in the operative position may vary as follows.

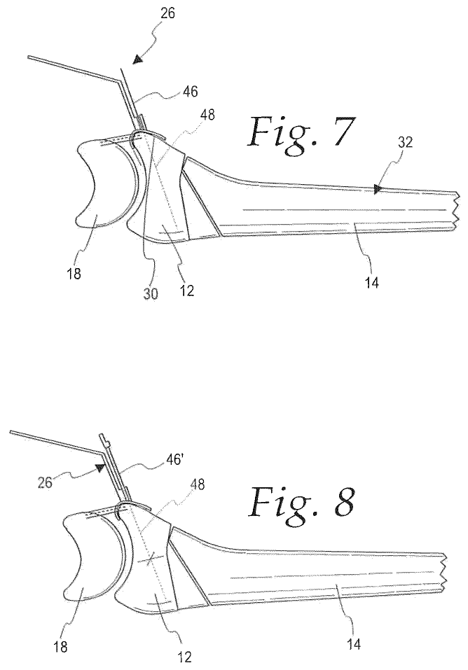

As shown in FIG. 7, once the surgeon has selected the desired angular orientation of the guide structure 26, a drilling tool 46 is directed into and through one of the guide passages 42—in this case the guide passage 42b—and into the first bone part 12 and can be used to further stabilize/anchor the guide structure 26. It should be understood that the discrete projecting referencing arms 38 can be constructed to engage the first bone part 12 so that they also stabilize, and potentially anchor, the guide structure 26, in one or more positions, alone or in conjunction with the drilling tool 46.

As used herein, “drilling tool” is intended to encompass a range of different components which may be pressed into the first bone part 12, as with a pin, or penetrate by rotational action, as with a boring component. Further, the drilling tool 46 may be a solid component or cannulated to facilitate guided movement relative to another component telescopingly engaged therewith.

Once the guide structure 26 is stabilized/anchored, as in FIG. 7, using the drilling tool 46 in the form of a provisional fixation pin directed through the guide passage 44b and into the first bone part 12, a drilling tool 46′ is advanced through the guide passages 44a, 44c to produce bores 48 in the first bone part 12 spaced from each other transversely to the bone length.

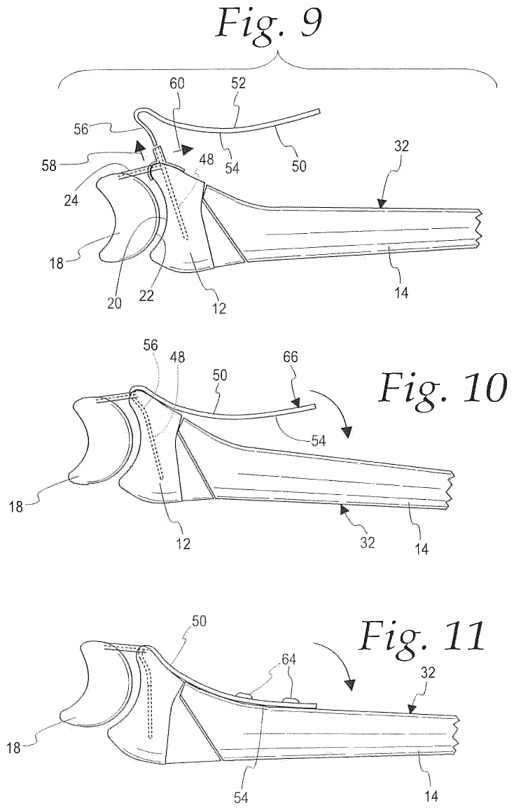

The method at this point changes depending upon the particular implant being utilized. The assignee herein offers an implant 50, as shown in FIGS. 9-11, which it identifies commercially as its “BUTTRESS PIN”. The implant 50 has a body 52 with a contoured surface 54 to generally match the configuration of, and bear against, a portion of the bone surface 32, and spaced legs/projecting parts 56 (one viewable in FIGS. 9-11) bent downwardly from the contoured surface 54.

The legs 56 and guide passages 42a, 42c are relatively spaced and dimensioned so that the legs 56 can be directed one each into the guide passages 42a, 42c and the preformed bores 48.

The surfaces bounding the guide passages 42a, 42c, in this exemplary form, extend only part way around the axes 44a, 44c so that with the implant 50 in the FIG. 9 position and the legs 56 penetrating the first bone part 12, the guide structure 26 may be raised slightly, as indicated by the arrow 58 in FIG. 9, to extract the discrete projecting referencing arms 38a, 38b from between the articular surface regions 20, 22 sufficiently that the ends 40a, 40b separate from the joint capsule 24, whereupon the guide structure 26 can be translated generally orthogonally to the lengths of the legs 56 either in the direction of the arrow 60, or oppositely, based upon the location of the interruption of the surfaces bounding the guide passages 42a, 42c, whereby the legs 56 are allowed to move out of their respective guide passage 42a, 42c to allow full separation of the guide structure 26 from the implant 50.

As shown in FIG. 10, the implant 50 is fully seated with respect to the first bone part 12. If the first bone part 12 is not malpositioned, the surface 54 may be against the bone surface 32, whereupon the fixation procedure can be completed by using one or multiple fasteners/screws 64.

Alternatively, as shown in FIG. 10, if the first bone part 12 is not properly oriented, so that the proximal region 66 of the implant is spaced from the surface 32, a force can be imparted to the implant 50, as by directly engaging and moving the implant 50, and/or the first bone part 12, to restore the first bone part 12 to the proper anatomical alignment, after which fixation to the stable part of the bone 10 can occur using the fasteners/screws 64.

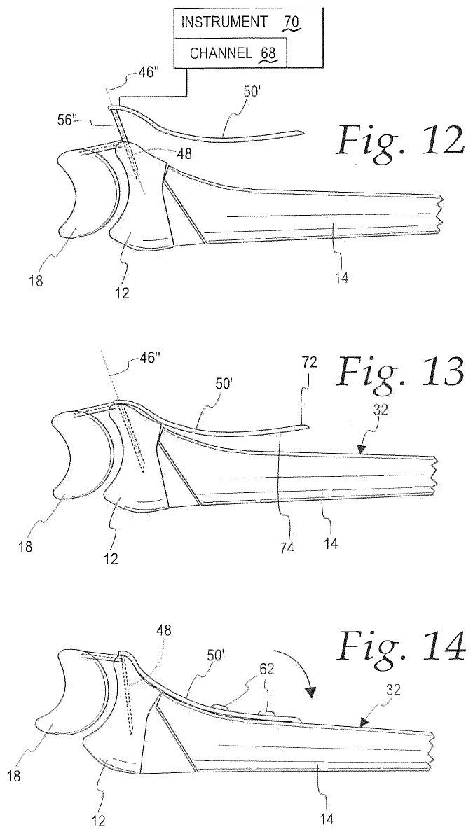

In FIGS. 12-14, a modified form of implant 50′ is utilized. The assignee herein offers this type of implant which it identifies as a “hook plate”.

To fix the implant 50′, the drilling tool 46′ is directed through guide passage 42b and left temporarily in place in the bone as shown in FIG. 8. Drilling tools 46″ can then be directed through the guide passages 42a, 42c (not shown in FIGS. 12-14) and into the first bone part 12 to create bores for projecting parts 56″, whereupon the guide structure 26 can be separated, in one form, by sliding the same lengthwise along the drilling tools 46″. The drilling tools 46″ can be directed either through holes on the implant 50′, as shown, or through a cannulated channel 68 on an instrument 70 attached to the implant 50′, as shown schematically as an alternative form in FIG. 12, and guide projecting parts 56″ into the bores and implant 50′ to coapt with bone parts 12 and 14.

The implant 50′ is then seated as shown in FIG. 13 and, if necessary, a proximal region 72 is pressed toward the bone surface 32 on the stable, second bone part 14 to place the bone engaging surface 74 against the bone surface 32 before completing the fixation process using fasteners/screws 62.

In FIGS. 16 and 17, a further modified form of guide structure is shown at 26′ to accommodate a further modified form of implant 50″, offered by the assignee herein commercially as its “VOLAR PLATE” with locked distal pegs/screws, having a corresponding projecting part 56″ angled differently on the implant 50″ than the projecting parts 56′ are on the implant 50′.

The body 28″ has an extension E with an additional block portion 36a″, spaced from a block portion 36″, that is substantially the same as the block portion 36. The block portions 36″, 36a″ might be formed as a single unit. Alternatively, the block portion 36″ could be eliminated. As shown in FIG. 16, in this embodiment the block portion 36″ and block portion 36a″ are formed as a unitary structure.

The block portion 36a″ defines a through guide passage 40a″ corresponding to the passages 40 and having an axis 44d at an angle α with respect to the axes 44″, corresponding to the axes 44.

The method can be performed in substantially the same manner, as described above, using the guide structure 26″ and implant 50″.

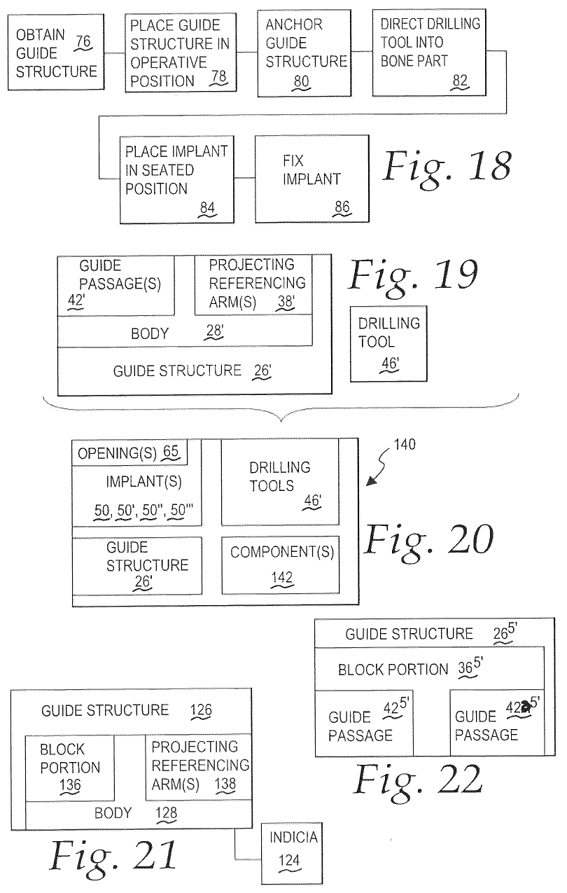

Accordingly, a method of fixing an implant, such as the implants 50, 50′, 50″ with respect to first and second bone parts can be performed as shown in flow diagram form in FIG. 18. The exemplary implant has a bone engaging surface and at least one projecting part.

As shown at block 76, a guide structure is obtained having a surface configured to bear against a surface on the first bone part, thereby establishing at least a preliminary referencing location RL. The engaging surface on the guide structure and first bone part may be with or without nominally matched contours-in the latter case potentially with one or more small area surfaces that abut. The guide structure has a body with at least a first guide passage with an axis.

As shown at block 78, the guide structure is placed in an operative position wherein the at least first discrete projecting referencing arm has penetrated the joint capsule and the surface on the guide structure is in confronting relationship with the surface region on the first bone part with the at least first discrete projecting referencing arm adjacent to the joint surface on the first bone part. The at least first discrete projecting referencing arm so situated can be abutted to the joint surface on the first bone part so as to thereby limit shifting of the guide structure relative to the first bone part lengthwise of the bone and thereby establish the aforementioned referencing location RL with respect to the joint surface on the first bone part. With the guide structure in the operative position, wherein the surface on the guide structure is in confronting relationship with the surface region on the first bone part, the axes of a guide passage on the guide structure intersect the first bone part at a predetermined location spaced from the referencing location RL lengthwise of the bone and at a predetermined angle with respect to the first bone part.

As shown at block 80, the guide structure is maintained in the operative position by being held or anchored thereon.

As shown at block 82, with the guide structure in the operative position, a drilling tool is directed guidingly through the first guide passage to form a first bore in the first bone part.

As shown at block 84, the implant is placed in a desired seated position. The one projecting part on the implant is then advanced into the first bore so that the bone engaging surface on the implant is in confronting relationship with the surface on the first bone part.

As shown at block 86, with the implant in the seated position, the implant is fixed to the second bone part.

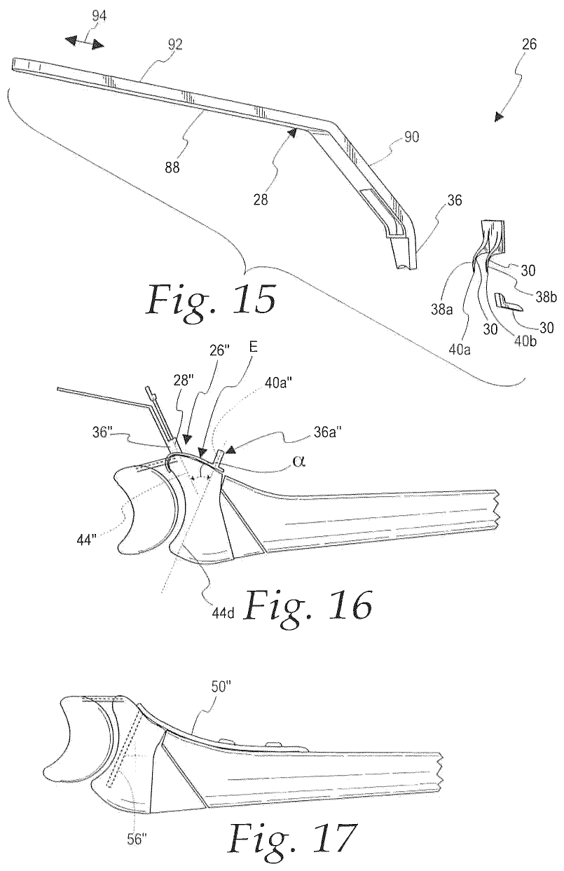

As seen clearly in FIGS. 4 and 15, the guide structure 26 has an elongate handle 88 projecting in cantilever fashion. The elongate handle 88 has an overall “L”/angular shape with a first leg 90 and a second leg 92. In the form depicted, the first leg 90 is connected to the block portion 36, with the second leg 92 extending from the first leg and defining a gripping length projecting in a line, indicated by the double-headed arrow 94, that is angled with respect to the axes 44 of the guide passages 42. The gripping handle 88 may be either fixed or releasably fixed to the block portion 36.

The gripping length is configured to be grasped by a user to facilitate holding and repositioning of the guide structure body 28 as procedures are being performed.

The gripping length may be oriented on the guide structure 26 to give a visual indication of the orientation of the particular implant subsequently fixed using the guide structure in the selected operative position. For example, the gripping length may align with the length of the second bone part corresponding to how the length of the implant in its seated position will align. This facilitates precise alignment of the guide structure in the operative position so that with the implant fixed, there is coaptation of the bone engaging surface on the implant along the coincident surface length on the second bone part on the bone 10 for which the implant is designed.

In an alternative form, as shown generically in FIG. 21, indicia 124, in any of potentially many different forms, may be incorporated to visually assist in arriving at: a) the desired orientation of an implant placed in a seated position using a guide structure 126, corresponding to the guide structure 26, in a selected operative position; and/or b) a desired location of an end of an implant fixed using the guide structure in the selected operative position. The generically depicted guide structure 126 has a body 128 with a block portion 136 with at least one projecting referencing arm 138. The indicia 124 might be dedicated structures or markings on the guide structure. For example, a discrete mark on the body 128 of the guide structure might identify a desired location of a distal end of an implant. An elongate bar 124″, as shown in FIG. 6, might indicate to a surgeon a resulting orientation of the length of an implant in relationship to the bone 10 by projecting in a direction parallel to the length of the implant, once seated.

The guide structure 26 is not limited to the configuration as shown in FIGS. 4-17. As shown schematically in FIG. 19, the basic guide structure 26′ may be usable to perform variations of the inventive method by having a body 28′ with as few as one guide passage 42′ and potentially more than three, and as few as one discrete projecting referencing arm 38′ with potentially more than the two shown. For example, the discrete projecting referencing arm(s) 38′ may be any configuration capable of penetrating a joint capsule 24 and potentially performing an anchoring function for the guide structure 26′ or alternatively stabilizing and performing a locating function while preventing shifting of the guide structure 26′ lengthwise of the bone 10, with anchoring additionally accomplished by one or more appropriate drilling tools 46′. In either case, the projecting referencing arms, once abutted to the first bone part 16, may establish the aforementioned referencing location RL. The guide structure may include a positioning element and may be with or without guide passages.

The discrete projecting referencing arms 38′ may be provided in shape and number to facilitate placement of the guide structure in its final operative position by guiding movement relative to the first bone part 12 around and along one, two, or three axes.

Using the basic concepts described above, different variations of the method can be performed using different drilling tools 46, 46′, different forms of the guide structure 26, 26′, and different implants 50, 50′, 50″.

The implants may have many other different forms with at least one projecting part either integrally formed with a part of the implant that defines the bone engaging surface, or fixedly attached thereto. As explained below, the inventive method can be practiced with implants having no projecting part.

In a further variation of the guide structure, the distance D in FIG. 6 may be variable. In the aforementioned form, as shown schematically in FIG. 21, the guide structure 126, corresponding to the guide structure 26, has a block portion 136 and at least one projecting referencing arm 138 mounted on a body 128 to be relatively movable in one, two, or three dimensions. In another variation, a secondary guide piece can be applied to create a different distance D.

As shown at FIG. 20, the method may further include providing a kit at 140 including at least one of: a) different forms of implant 50, 50′, 50″, 50′″; b) different forms of guide structure 26′; c) different forms of drilling tools 46′; and d) additional miscellaneous components 142 usable to perform the methods described herein.

In another variation, as shown in FIG. 22, a block portion 365′ has guide passages 42⁵′, 42a⁵′ spaced from each other lengthwise of the bone 10 with the associated guide structure 26⁵′ in its operative position. Accordingly, the axes of the guide passages 42⁵′, 42a⁵′ intersect the first bone part 12 at different distances from the referencing location RL, as to accommodate different bone configurations, including one with different sizes of rims/edges 25.

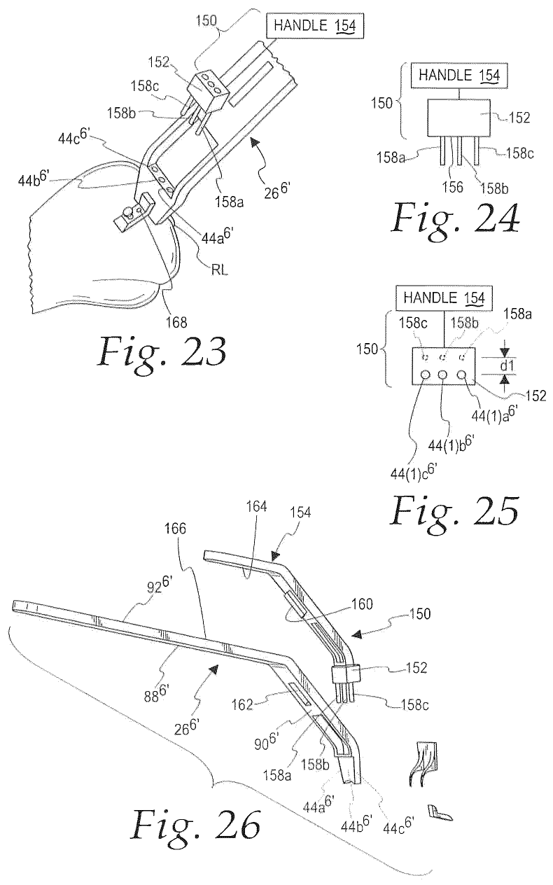

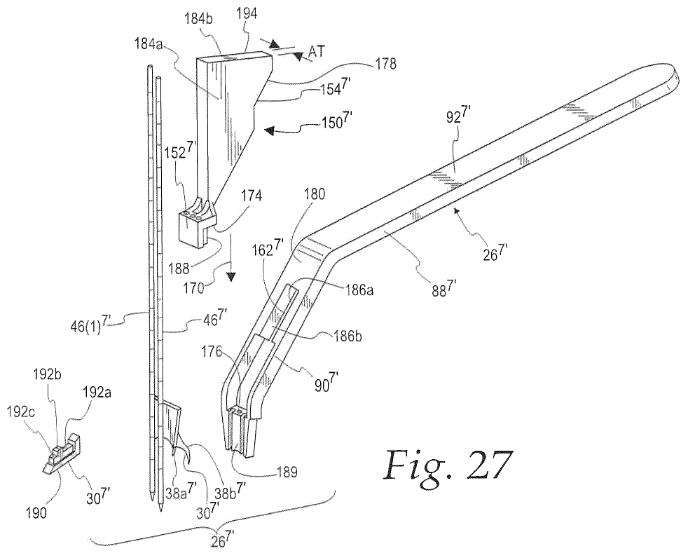

The ability to adjust the distance D in FIG. 6 has been described generically above, as with specific reference to the schematic depiction in FIG. 21. An exemplary form of the secondary guide piece referenced above is shown in FIGS. 23-26. As shown in these Figures, an adaptor 150 is utilized in conjunction with a guide structure 26⁶′, corresponding to the guide structure 26 as shown in FIG. 15, with certain modifications explained below.

The adaptor 150 consists of a body 152 having an associated handle 154.

The body has a bottom surface 156 from which three alignment components depend in substantially parallel lines. The alignment components 158a, 158b, 158c are configured and arranged to be simultaneously directed into the guide passages 44a⁶′, 44b⁶′, 44c⁶′, successively, on the guide assembly 26⁶′. By advancing the alignment components 158a, 158b, 158c guidingly within respective guide passages 44a⁶′, 44b⁶′, 44c⁶′, the adaptor 150 can be placed into an assembled position wherein an elongate rib 160 on the underside of the handle 154 extends into, and makes a keyed connection with, a complementarily-shaped slot 162 on the guide structure 26⁶′.

With the adaptor 150 in the assembled position, an underside region 164 thereof either abuts to or is placed in close proximity to an oppositely facing surface region 166 on the guide structure 26⁶′.

In this embodiment, the guide structure 26⁶′ has an elongate handle 88⁶″ with first and second legs 90⁶′, 92⁶′ corresponding to the legs 90, 92 on the handle 88⁶′ of the guide structure 26. The handle 154 on the adaptor 150 has a bent shape generally complementary to the shape of the handle 88⁶′ whereupon with the adaptor 150 in the assembled position, the surgeon can wrap his/her hand around the stacked arrangement of the handles 88⁶′, 154.

The body 152 on the adaptor 150 has additional guide passages 44(1)a⁶′, 44(1)b⁶′, 44(1)c⁶′, in the embodiment shown each spaced a distance d1 respectively from the axes of the alignment components 158a, 158b, 158c, which in turn successively coincide with the axes of the guide passages 44a⁶′, 44b⁶′, 44c⁶′ with the adaptor 150 in the assembled position.

With the adaptor 150 in the assembled position, the guide passages 44(1)a⁶′, 44(1)b⁶′, 44(1)c⁶′ are spaced further from the referencing location RL than the guide passages 44a⁶′, 44b⁶′, 44c⁶′.

The distance d1 may be 2-3 millimeters as to accommodate different bone lengths. However, the distance d1 may be shorter than 2 millimeters or greater than 3 millimeters.

The guide passages 44(1)a⁶′, 44(1)b⁶′, 44(1)c⁶′ are usable in the same manner as described hereinabove for the guide passages 44a, 44b, 44c, to which the guide passages 44(1)a⁶′, 44(1)b⁶′, 44(1)c⁶′ successively correspond.

The guide passages 44(1)a⁶″, 44(1)b⁶′, 44(1)c⁶′ may have a non-parallel relationship to the guide passages 44a⁶′, 44b⁶′, 44c⁶′, depending upon the site condition and the desired trajectory, which may vary based upon the particular implant being utilized.

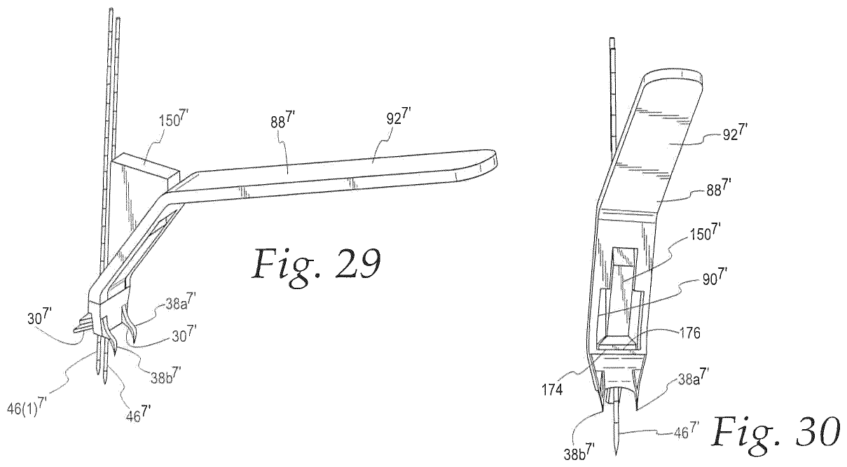

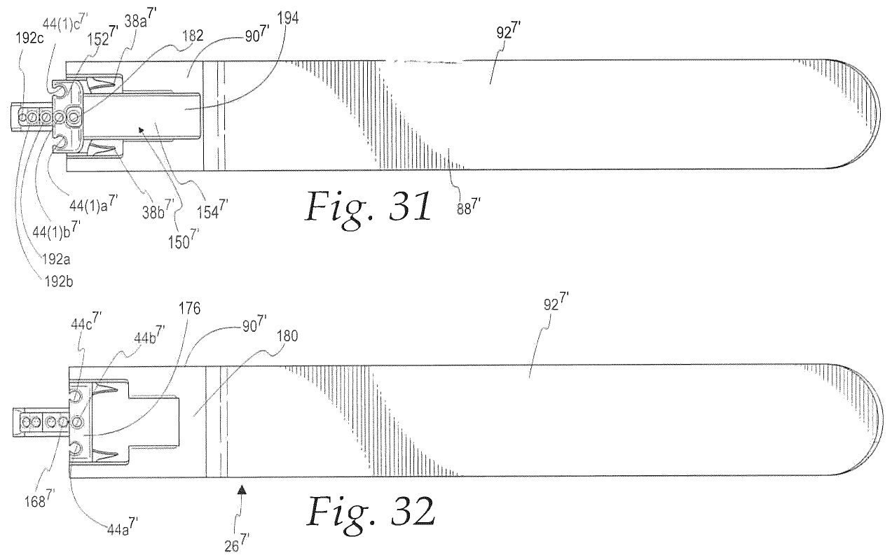

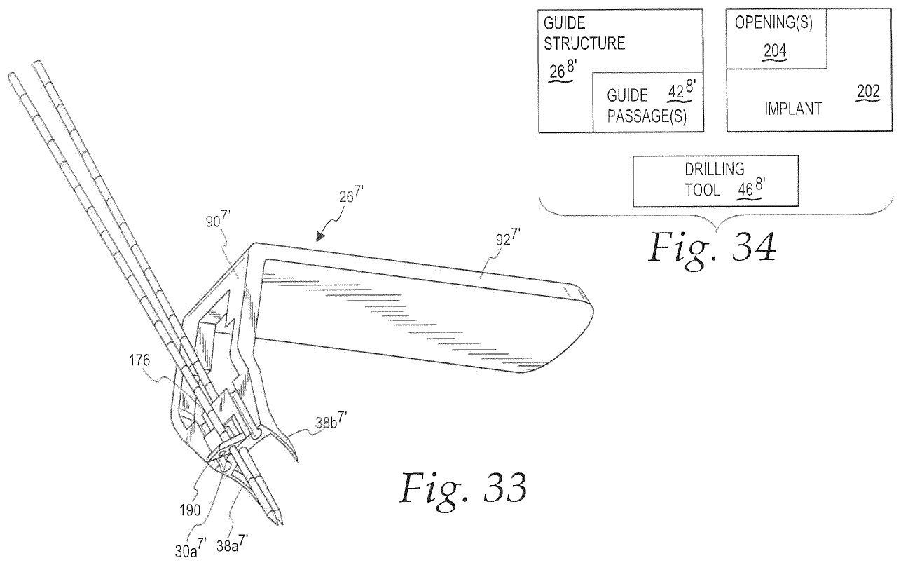

In FIGS. 27-33, another form of adaptor is shown at 150⁷′ used in conjunction with a modified form of guide structure 26⁷′.

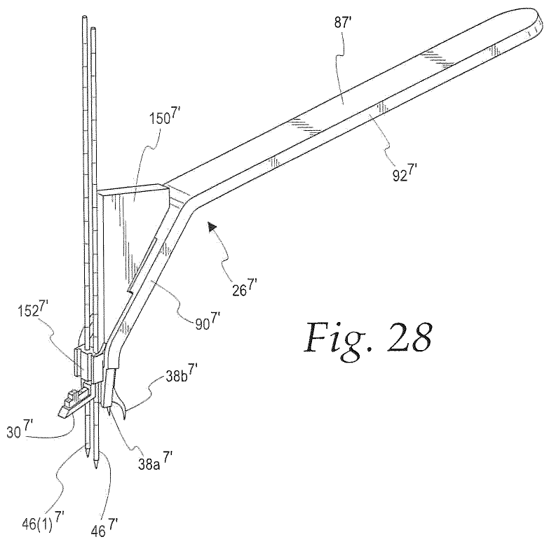

The adaptor 150⁷ is configured to be press fit into an assembled position on the guide structure 26⁷′ as shown in FIGS. 28-31.

The guide structure 26⁷′ has an elongate handle 88⁷′ with first and second legs 90⁷′, 92⁷′ cooperating to produce a generally “L”/angular shape similar to that for the handles 88, 88⁶′, described above.

The guide structure 26⁷′ has guide passages 44a⁷′, 44b⁷′, 44c⁷′ corresponding to those 44a⁶′, 44b⁶′, 44c⁶′ and 44a, 44b, 44c in other embodiments, described above.

A slot 162⁷′ is formed through the leg 90⁷′ to receive a part of the adaptor 150⁷′. With the adaptor 150⁷′ aligned over the slot 162⁷′, as shown in FIGS. 27, the adaptor can be advanced downwardly so that a thickness AT of a main, graspable portion 154⁷′ of the adaptor 150⁷ is guided within the slot 162⁷′ as the adaptor 150⁷′ moves in the direction of the arrow 170 towards the assembled position in FIGS. 28-31.

The adaptor 150⁷′ has a body 152⁷′ that depends from the graspable portion 154⁷′ and is offset therefrom. The body 152⁷′ on the adaptor 150⁷′ has guide passages 44(1)a⁷′, 44(1)b⁷′, 44(1)c⁷′ corresponding to the guide passages 44(1)a⁶′, 44(1)b⁶′, 44(1)c⁶′ on the body 152 of the adaptor 150, described above.

Where the graspable portion 154⁷′ of the adaptor 150⁷′ transitions to the body 152⁷′, a downwardly facing step 174 is defined. With the adaptor 150⁷′ in the assembled position, the step 174 abuts an upwardly facing surface 176 on the guide structure 26⁷′ through which the openings 44a⁷′, 44b⁷′, 44c⁷′ extend.

With the adaptor 150⁷′ in the assembled position, an angled, downwardly facing surface 178 on the adaptor 150⁷′ abuts to a confronting surface portion 180 on the leg 90⁷′ on the guide structure 26⁷′.

The adaptor 150⁷′ has an additional alignment passage 182 which, with the adaptor 150⁷′ in the assembled position, axially aligns with the guide passage 44b7′ on the guide structure 26⁷′. With the adaptor 150⁷′ in the assembled position, a drilling/alignment tool 46⁷′ can be directed through the alignment passage 182 and into the guide passage 44b⁷′, whereby the adaptor 150⁷′ is consistently blocked in a predetermined assembled position.

With this configuration, there are a series of connectors cooperating between the adaptor 150⁷′ and the guide structure 26⁷′ that both guide the adaptor 150⁷′ towards its assembled position and maintain the adaptor 150⁷′ stably and consistently in a predetermined assembled position.

For example, the opposite side surfaces 184a, 184b on the graspable portion 154⁷′ respectively cooperate with surfaces 186a, 186b, bounding the slot 162⁷′, to both guide translational movement of the adaptor 150⁷′ towards its assembled position and key the adaptor 150⁷′ against turning around a vertical axis relative to the leg 90⁷′.

The step 174 and surface 176 function as cooperating connectors, as do confronting surfaces 188, 189, respectively on the body 152⁷′ and the guide structure 26⁷′.

Further, the locating/drilling tool 46⁷′ cooperates between the adaptor 150⁷′ and the guide structure 26⁷′ to maintain the connection therebetween with the adaptor 150⁷′ in the assembled position.

The guide structure 26⁷′ has an opening 168⁷′ corresponding to the opening 168 on the guide structure 26⁶′, which aligns with the guide passage 44(1)b⁷′ on the adaptor 150⁷′ to accept a locating/drilling tool 46(1)⁷′ directed through the guide passage 44(1)b⁷′ on the adaptor 150⁷′ with the adaptor 150⁷′ in the assembled position.

With the described construction, the adaptor 150⁷ can be press fit into the assembled position. The locating/drilling tool 46⁷′ can be directed through the guide passage 44b⁷′ before or after the adaptor 150⁷′ is placed into the assembled position. In the former case, the locating/drilling tool 46⁷′ also guides the adaptor 150⁷′ consistently into the assembled position.

A projecting piece 190, corresponding generally in function to the extension E on the support 28″ as shown in FIG. 16, defines in conjunction with the projecting referencing arms 38a⁷′, 38b⁷′ a contoured surface 30⁷′ to be borne against, or sit above, the surface region 34. The projecting piece 190 may have one or more additional guide passages 192a, 192b, 192c formed therethrough to accept drilling tools. As depicted, the projecting piece 190 is integrally formed with the leg 90⁷′ at the surface 189.

While the connectors cooperating between the adaptor 150⁷′ and the guide structure 26⁷′ may adequately maintain the adaptor 150⁷′ in its assembled position, the adaptor 150⁷′ and handle 88⁷′ are configured so that with the surgeon's hand grasping the leg 92⁷′, his/her thumb might be pressed against an upwardly facing surface 194 on the adaptor 150⁷′ to avoid any upward shifting thereof as a procedure is being performed.

The details of the guide structure 26⁷ shown in FIGS. 27-33, such as those for the depicted referencing arms 38a⁷′, 38b⁷′, may be incorporated into all embodiments described herein.

Different types of adaptors might be provided to have different arrangements of guide passages in terms of spacing from each other and the referencing location RL, axial trajectory, etc.

It should also be noted that a particular adaptor may have only a single alignment component to cooperate with a single guide passage, with alignment of the adaptor in the assembled position maintained by additional structure, such as the aforementioned rib 160 and slot 162, or other readily devisable keying structure.

Many other structures might be devised to allow an adaptor to be connected to a guide structure as described herein to be utilized in conjunction therewith and provide a different arrangement of one or more guide passages usable to perform procedures as described above.

A press fit arrangement may be utilized with or without a handle corresponding to the handle 154. A frictionally held or snap connected adaptor may be utilized.

A single adaptor may have any number of guide passages spaced from each other in different relationships and with different trajectories.

Preferably, with the adaptor in the assembled position, connectors on the adaptor and guide structure cooperate to consistently maintain a preselected relationship between the adaptor and guide structure.

It is also contemplated that the foundational guide structure may have any arrangement of guide passages, spaced different distances from the referencing location RL and each other. The axes of the guide passages may have different trajectories, whereby a single guide structure might have substantial versatility—usable at different sites and with different implants.

It is noted that with the depicted guide structure 26⁶′, an opening 168 is required to be formed therethrough the accommodate a particular drilling tool that may be directed through the guide passage 44(1)b⁶′.

In all embodiments, the guide structure may be designed with a guide passage that is situated to guide a drilling tool to form a locating bore in the first bone part. With the drilling tool remaining in the locating bore, the implant can be lowered to thereby direct an end of the drilling tool into an opening through the implant whereupon the implant can be advanced guidingly along the drilling tool towards its predetermined, desired, seated position. The implant opening may be a dedicated opening or an opening that is provided on the particular implant for reasons other than to guide a drilling tool.

The implant opening may be a small opening to receive a drilling tool in the form of a conventional K-wire, such as the at least one opening 65, depicted schematically in FIG. 20, to encompass one or more dedicated openings or one or more openings existing for other reasons, such as to facilitate fixing of the implant. The K-wire in the locating bore establishes the location at which a single, or separate, bores will be formed, as for projecting parts on the implant using the guide structure. Further, the K-wire in the locating bore guides the implant so that the projecting parts of the implant will be located at a desired location and/or angle in reference to the joint surface.

In one exemplary form, the K-wire producing the locating bore would be directed through the center guide passage 44b in each of the above-described embodiments or the center guide passage 44(1)b on the adaptors 150.

The same concepts can be used to facilitate placement of an implant without any projection or with a projection that is coupled to the implant. For example, the same concepts can be used to optimally locate an implant such as the assignee's VOLAR BEARING PLATE™, representative of implants that can be applied without any projection or have projections coupled to the implant after application, shown schematically at 202 in FIG. 34. The implant has at least one opening 204 therethrough, which may be an opening to accept a fastener, an opening generally smaller in diameter than one designed for accepting a fastener, or another type of opening.

A guide structure 26⁸′, as shown schematically in FIG. 34, has at least one guide passage 42⁸′ to guide a drilling tool 46⁸′ along an axis. The guide structure 26⁸′ may be the same as any of the guide structures described above, or may have a different form including, but not limited to, one that will accept and dictate a bone entry location of a drilling tool without performing the translational guiding function. With potentially a single guide passage 42⁸′, the guide structure can be placed in an operative position whereupon the drilling tool 46⁸′ can be directed guidingly into a first bone part to form a locating bore. With the drilling tool 46⁸′ remaining in the locating bore on the first bone part, the implant can be situated to align the implant opening 204 over an end of the drilling tool 46⁸′. By thereafter lowering the implant, the implant will be guided by the drilling tool 46⁸′ to a predetermined, desired seated position against the first bone part. The implant could be designed to allow the implant to be shifted orthogonally to the length of the drilling tool 46₈′ to admit the drilling tool 46₈′ into the opening 204.

The guide structure 26⁸′ is designed so that the drilling tool 46⁸′ will cause the implant 202 to be consistently optimally aligned lengthwise of the bone. By providing multiple openings 204, each cooperating with a drilling tool 46⁸′ guidingly advanced into the first bone part in like fashion, the orientation of the implant 202 can be consistently established in multiple directions.

It is contemplated that the guide structure can be used in many different forms and ways to align an implant in a predetermined, desired, seated position in relationship to the first bone part. This alignment may be lengthwise of the bone and/or angularly around the bone length.

In one form, the guide structure in the operative position is used to facilitate alignment of an implant by simply defining a reference structure. With the guide structure in the operative position, an implant may be abutted to a part of the guide structure to thereby establish the desired lengthwise position for the implant relative to the joint referencing location.

Alternatively, structure and/or indicia may be provided on the operatively positioned guide structure to allow the surgeon to visually determine the predetermined, desired alignment for the implant.

The interaction between the guide structure and an implant, during placement thereof, may involve the use of one or more drilling tools or may take place without any drilling tool.

The guide structure may also be used, as described above, to align implants with one or more projecting parts by facilitating formation of strategically placed bores in the first bone part to accept the implant projecting part(s).

Further, the guide passage in the guide structure need not be formed to controllably guide translation of a drilling tool. Rather, the guide passage may be formed as a locating opening simply to permit strategic bore formation in the first bone part that will align with an implant opening, as to receive a fastener or having another function. The procedure with this design may or may not involve use of a drilling tool, or another component capable of penetrating the first bone part.

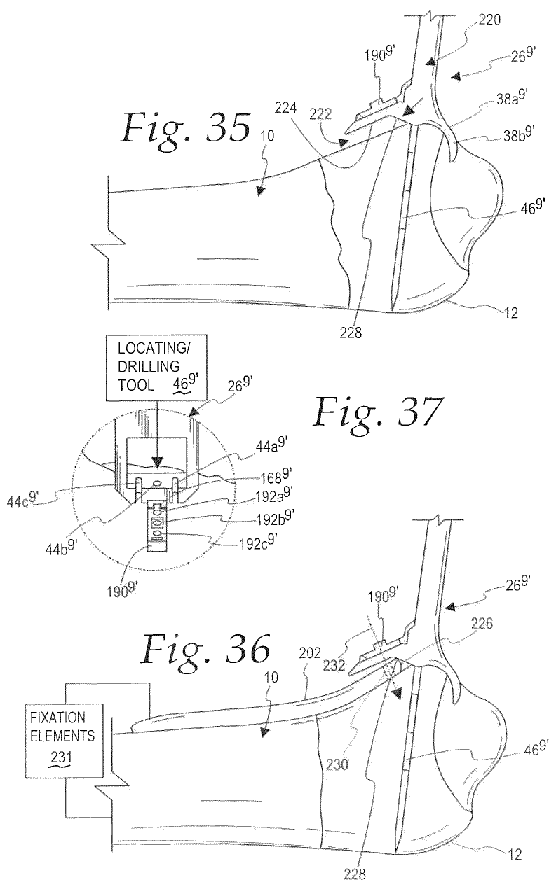

A further modified form of guide structure is shown at 26⁹′ in FIGS. 35-37. The guide structure 26⁹′ has the general configuration of the guide structure 26⁷′, including a projecting piece 190⁹′ corresponding to the projecting piece 190. However, a body 220, including integrally formed referencing arms 38a⁹′, 38b⁹′ and the projecting piece 190⁹′, is configured so that with the guide structure 26⁹′ in the operative position on the bone 10, including the first bone part 12, a controlled gap 222 is maintained between an underside 224 of the extension 190⁹′ and the first bone part 12. Guide passages 44a⁹′, 44b⁹′, 44c⁹′ are provided as on the guide structure 26⁷′. As depicted, a locating/drilling tool 46⁹′ is directed through the guide passage 44b⁹′ and into the first bone part 12 to maintain the operative position in FIGS. 35-37.

Without limitation, the guide structure 26⁹′ accommodates the implants 202, described generally above and sold by the assignee as its VOLAR FIXED ANGLE PLATE™ and VOLAR BEARING PLATE™. The implant 202 can be directed into the gap 222 and advanced therein until a leading end 226 thereof abuts a stop surface 228 on the guide structure 26⁹′ bounding the gap 222. Alternatively, a drilling tool may be inserted into one of the guide openings 192a⁹′, 192b⁹′, 192c⁹′ to perform the function of the stop surface 228. This allows for consistent placement of the implant 202 with respect to the bone 10 in the aforementioned desired seated position for the implant 202 at a specific distance from the joint referencing location. With the implant 202 in the desired seated position, one of the guide openings 168⁹′, 192a⁹′, 192b⁹′, 192c⁹′ on the projecting piece 190⁹′ may align with an opening 230 through the implant 202. An appropriate fixation element 231 can be directed along the axis 232 through the extension 190⁹′ and the implant 202 and into the first bone part 12. Additional fixation elements 231 can be used to finally fix the implant 202 in the desired seated position by being directed into the bone parts 12, 14. The designs of the guide structure 26⁹′ and implant 202 can be coordinated so that different guide openings 168⁹′, 192a⁹′, 192b⁹′, 192c⁹′ can be used to accommodate different constructions for the implant 202.

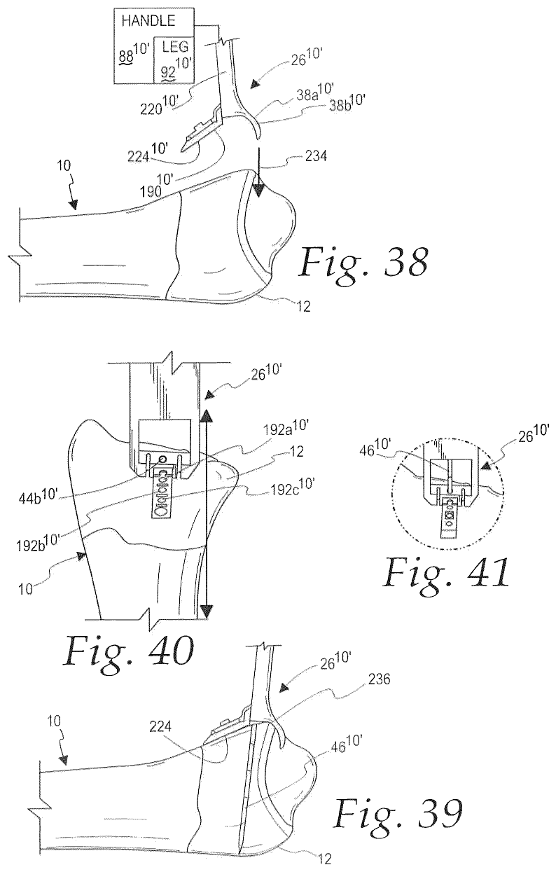

In FIGS. 38-41, a further modified form of guide structure is shown at 26¹⁰′. This guide structure 26¹⁰′ will be used as an exemplary form in describing in further detail the controlled placement of each of different forms of implant, described above, in a desired seated position therefor. The guide structure 26¹⁰′ is substantially the same as the guide structure 26⁹′, with the exception that in the operative position therefor, the underside 224¹⁰′ abuts, or is in close proximity, to the bone 10, as opposed to maintaining the aforementioned gap 222.

The guide structure 26¹⁰′ has a body 220¹⁰′ with integrally formed referencing arms 38a¹⁰′, 38b¹⁰′ and a projecting piece 190¹⁰′. A handle 88¹⁰′ on the body 220¹⁰′ has an elongate leg 92¹⁰′ with a length that is aligned with the length of the bone 10, to facilitate placement of the guide structure 26¹⁰′ consistently in a desired operative position, as depicted in FIGS. 39-41.

With the guide structure 26¹⁰′ aligned in a starting position as shown in FIG. 38, with the handle 88¹⁰′ in most applications being aligned with the length of the bone 10, the referencing arms 38a¹⁰′, 38b¹⁰′ can be directed through the joint capsule (not shown) in the direction indicated by the arrow 234.

At an exemplary radiocarpal joint, the guide passage 44b¹⁰′ is aligned to generally match the central axis of the “teardrop” 236. To maintain this operative position, a locating/drilling tool 46¹⁰′, as in the form of a K-wire, is directed through the guide passage 44b¹⁰′ and into the first bone part 12.

With the guide structure 26¹⁰′ in the operative position of FIGS. 39-41, different forms of implant can be fixed in a desired seated position, as described hereinbelow.

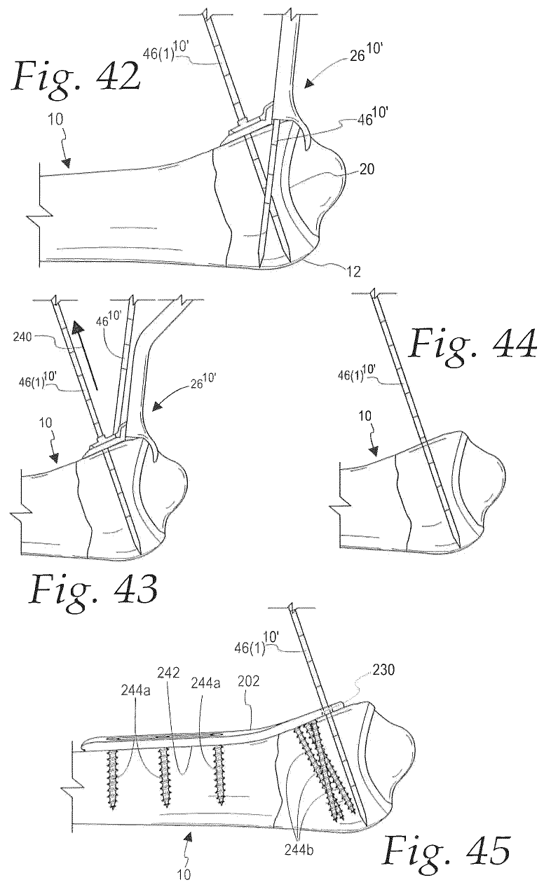

Referring to FIGS. 42-45, the operatively positioned guide structure 26¹⁰′ will be described as used to facilitate alignment of the VOLAR FIXED ANGLE PLATE and VOLAR BEARING PLATE implants 202 in desired seated positions, in a different manner than described above, as shown in FIG. 45.

A locating/drilling tool 46(1)¹⁰′ is directed through the guide passage 192b¹⁰′ (see FIG. 38) and into the first bone part 12, as seen in FIG. 42. The locating/drilling tool 46(1)¹⁰′ may be a K-wire. The relationship of the locating/drilling tool 46(1)¹⁰′ can be determined through an x-ray. If it is desired that the locating/drilling tool 46(1)¹⁰′ be closer to or further away from the joint surface 20, the locating/drilling tool 46(1)¹⁰′ can be removed and inserted through the guide passages 192a¹⁰′, 192c¹⁰′, respectively, and into the bone part 12.

The locating/drilling tool 46¹⁰′ can then be withdrawn and the guide structure 26¹⁰′ slid along the locating/drilling tool 46(1)¹⁰′, as indicated by the arrow 240, to allow separation of the guide structure 26¹⁰′ from the bone 10.

The end of the locating/drilling tool 46(1)¹⁰′ can then be directed through the opening 230, and the implant 202 thereafter guided along the locating/drilling tool 46(1)¹⁰′ until the underside/bone-engaging surface 242 on the implant 202 is placed conformingly against the exposed surface of the bone 10.

Fixation components 244a, 244b, in the conventional form of locking pegs and bone screws, are used to effect final fixation.

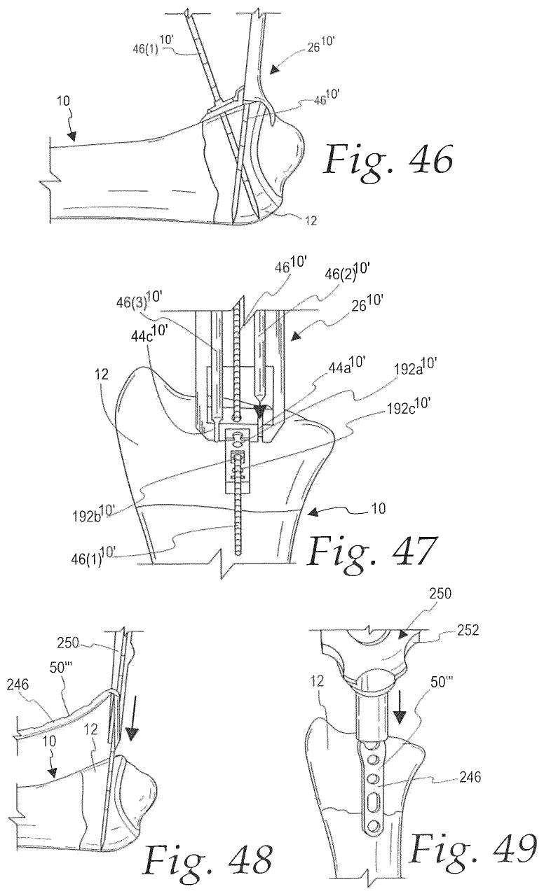

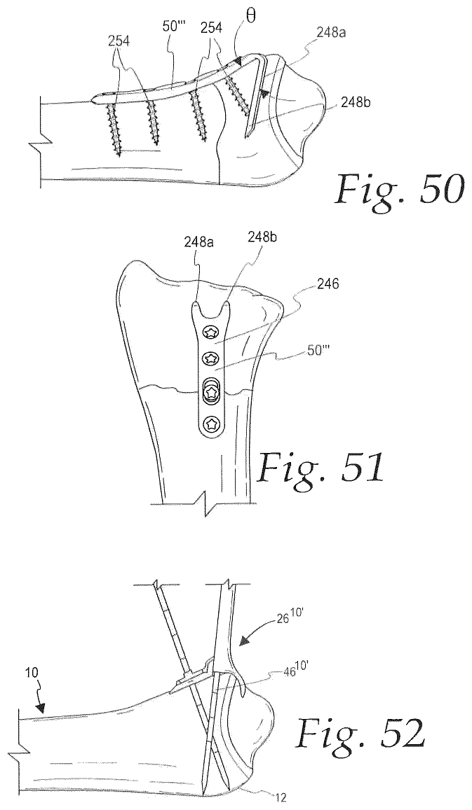

In FIGS. 46-51, the guide structure 26¹⁰′ is shown utilized to facilitate alignment of an implant 50′″ in the form of what the assignee identifies as its VOLAR HOOK PLATE™, having a main body 246 with two hook ends 248a, 248b projecting at an angle to the length of the body 246 so as to define an acute angle θ therebetween. Additional details of this VOLAR HOOK PLATE™ structure are disclosed in U.S. Pat. No. 8,821,508, the disclosure of which is incorporated herein by reference.

The guide structure 26¹⁰′ is placed in the operative position as shown in FIG. 46, which corresponds to the position shown in FIG. 42, and may be maintained in the operative position by the locating/drilling tool 46(1)¹⁰′ as also shown in FIG. 42—extending through the appropriate guide passage 192a¹⁰′, 192b¹⁰′, 192c¹⁰′. Placement in the passage 192b¹⁰′ also anticipates location of the distal fixed angle peg/end on the implant and can be used by the surgeon to confirm implant location.

Locating/drilling tools 46(2)¹⁰′ and 46(3)¹⁰′ are respectively directed through guide passages 44a¹⁰′, 44c¹⁰′ to form spaced bores in the first bone part 12. The locating/drilling tools 46(1)¹⁰′, 46(2)¹⁰′, 46(3)¹⁰′ are then separated, whereupon the guide structure 26¹⁰′ can be slid lengthwise off of the remaining locating/drilling tool 46¹⁰′.

The VOLAR HOOK PLATE™ implant 50′″ can then be engaged with an inserter 250, such as disclosed in U.S. Pat. Nos. 11,324,539 and 11,490,906, the disclosure of which is incorporated herein by reference. The locating/drilling tool 46¹⁰′ can then be directed into the cannulation in the inserter 250, whereupon a drive component 252 on the inserter 250 is impacted to seat the hooked ends 248a, 248b, one each in the bores defined by the locating/drilling tools 46(2)¹⁰′, 46(3)¹⁰′. With the hooked ends 248 fully seated, the VOLAR HOOK PLATE™ implant 50′″ is fixed in the desired seated position through fixation components 254, in the form of conventional locking pegs and screws.

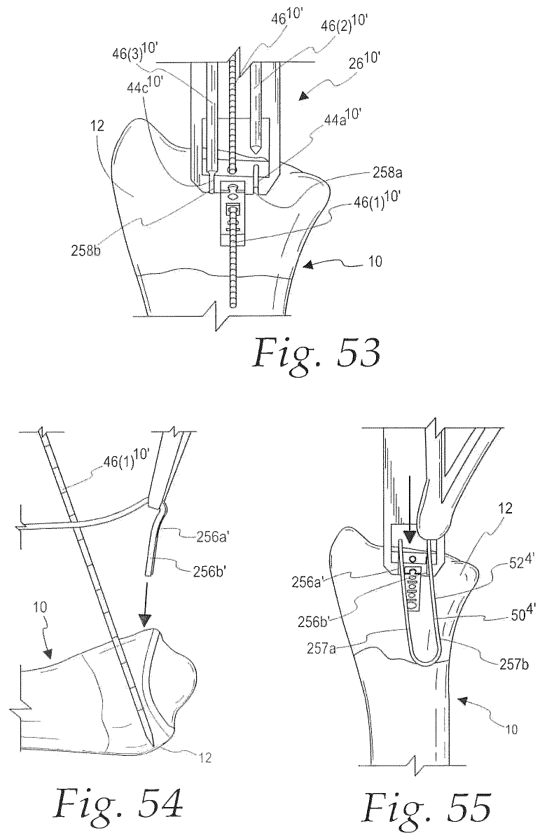

Additional details regarding placement of an implant of the type of the implant 50, described with respect to FIGS. 9-11, are described hereinbelow using the guide structure 26¹⁰′, in FIGS. 52-57. The depicted form is what the assignee offers as its “VOLAR BUTTRESS PIN”™.

FIGS. 52-55 show the guide structure 26¹⁰′ in the operative position, corresponding to the position shown in FIGS. 46 and 47, above in preparation for facilitating alignment of an implant 50⁴′ in a desired seated position. Locating/drilling tools 46(2)¹⁰′ and 46(3)¹⁰ are guidingly directed into the first bone part 12 in the same manner as described with respect to FIG. 45.

Once bores are formed by the locating/drilling tools 46(2)¹⁰′ and 46(3)¹⁰′, these locating/drilling tools are fully separated. This leaves the locating/drilling tool 46(1)¹⁰′ maintaining/stabilizing the guide structure 26¹⁰′ in its operative position.

The body 52⁴′ of the implant 50⁴′ has a “U” shape defined by a continuous wire form, which also defines the angled legs 256a′, 256b′. The implant 50⁴′ is moved to the preliminary FIG. 54 position wherein legs 257a, 257b defining the body 52⁴′ straddle the locating/drilling tool 46(1)¹⁰′, which allows the legs 256a′, 256b′ to align one each with the bores formed by the locating/drilling tools 46(2)¹⁰′ and 46(3)¹⁰′. With the guide structure 26¹⁰′ maintained in the operative position, the guide passages 44a¹⁰′, 44c¹⁰′ guide the legs 256a′, 256b′ into their respective bores in the first bone part 12.

The guide passages 44a¹⁰′, 44c¹⁰′ respectively have discrete circumferential slots 258a, 258b which allow the guide structure 26¹⁰′ to be separated from the implanted legs 256 by shifting the guide structure 26¹⁰′ transversely to the length of the legs 256 through the slots 258a, 258b.

The legs 256 can be driven into place until the body 52⁴′ conformingly abuts to, or comes into close proximity with, the bone 10.

In this embodiment, plates 260a, 260b are connected to the body 52⁴′ as described in U.S. Pat. No. 7,942,877, the disclosure of which is incorporated herein by reference. Appropriate fasteners 262 are directed through the plates 260a, 260b and into the bone 10 to fix the body 52⁴′ against the bone 10. Alternatively, the implants are fixed directly as disclosed in U.S. application Ser. No. 17/152,253, the disclosure of which is incorporated herein by reference.

It should be understood that many different variations of the invention are contemplated, with the above embodiments being exemplary in nature only. The guide structures can be used in many different manners to facilitate alignment of an implant in a desired seated position.

As just one additional example, a modified form of the inventive guide structure is shown at 26¹¹′ in FIG. 58. As depicted, potentially a single projecting referencing arm 38¹¹′ may be utilized having a curved portion extending up to a free end 264 that is abutted to a bone to establish a reference location RL. The free end 264 acts as a fulcrum which allows a surgeon to reorient the guide structure through a handle 88¹¹′. As depicted, the free end 264 may be the only “surface” that contacts the bone at the joint. A projecting portion 266 has at least one guide passage 268, corresponding to the aforementioned guide passages 44. The projecting portion 266 may be unsupported as the guide passage(s) 268 is utilized to control entry of the locating/drilling tool 46 and/or part of an implant.

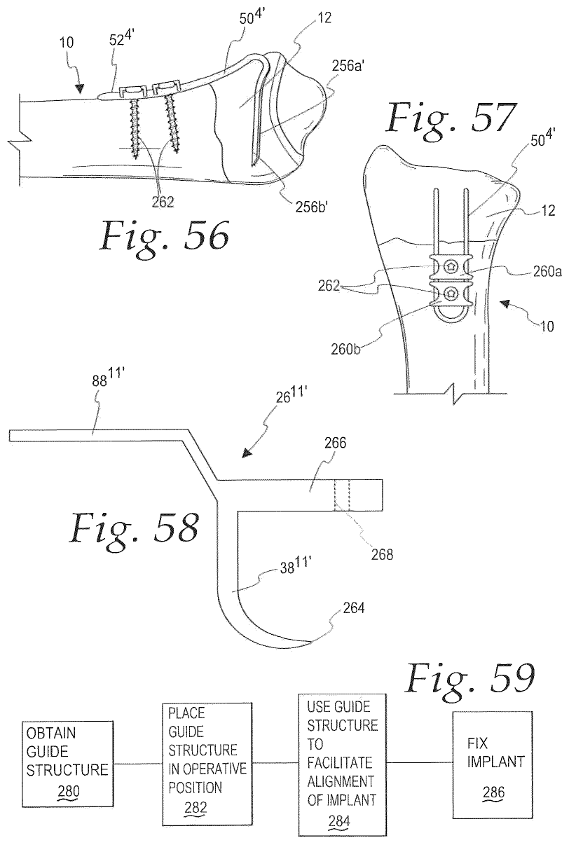

Further, the same basic configuration as shown in FIG. 56 with a single referencing arm may be used, having the general configuration of the referencing arm 38′″ or any of the referencing arms 38, described above, without the need for a guide passage such as the guide passage 268. This guide structure may incorporate any of those capabilities, described above, to facilitate alignment of an implant in the desired seated position.

It should be understood that the method can be practiced at any joint location on the human body. As just examples, but without any limitation, exemplary implants and procedures that can be used/performed using the inventive method are described on the assignee's website trimedortho.com. The guide structure can be strategically designed in different forms to facilitate optimal placement of different forms of implant at different joint locations.

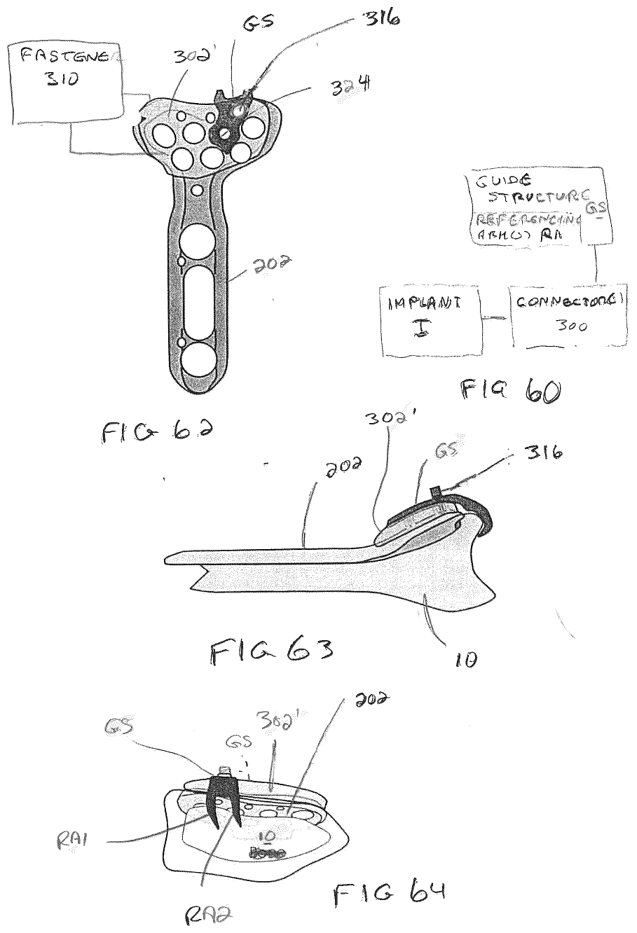

FIG. 59 includes a further flow diagram representation of a method of fixing an implant with respect to first and second bone parts at a joint with an adjacent bone, with the first and second bone parts making up one bone with a length.

As shown at block 280, a guide structure is obtained having a body with at least a first discrete projecting referencing arm.

As shown at block 282, the guide structure is placed in an operative position by causing the at least first discrete projecting referencing arm to penetrate a joint capsule between the first bone part and the adjacent bone and position adjacent a joint surface on the first bone part to abut the joint surface on the first bone part so as to thereby limit shifting of the guide structure relative to the first bone part at least lengthwise of the one bone and establish a referencing location with respect to the joint surface on the first bone part.

As shown at block 284, with the guide structure in the operative position, the guide structure is used to facilitate alignment of the implant in a desired seated position wherein a bone engaging surface on the implant is in confronting relationship with a surface on the one bone.

As shown at block 286, the implant is fixed in the desired seated position.

The generic depiction in FIG. 59 is intended to encompass virtually an unlimited number of different ways that the inventive guide structure might be utilized to facilitate placement of different forms of implant in a desired seated position.

In FIG. 60, an implant I is shown having a guide structure GS with at least one referencing arm RA, that is operatively positioned on, and releasably joined to, the implant I using at least one connector 300. The generic showing of the implant I and guide structure GS is intended to encompass all versions of implants hereinabove described, as well as others known, or that become known, to those skilled in the art, as well as, without limitation, those guide structures likewise hereinabove described as exemplary forms.

With this construction, rather than maintaining the implant and guide structure as separate components during placement of the implant, the guide structure GS is releasably joined to the implant I through the connector(s) 300 during the placement process. The guide structure GS performs the same function as described for the guide structures described hereinabove, with the only exception being that the guide structure GS and implant I are handled as a unit, with the guide structure ultimately separated, preferably after the implant is placed in the desired seated position, and potentially after the fixation of the implant I is carried out.

The schematic depiction in FIG. 60 is intended to encompass any structure wherein a guide structure GS is joined to an implant I during placement of the implant I, and subsequently separated. Exemplary forms of guide structure GS and implant I will be described hereinbelow, with it being understood that these exemplary forms should not be interpreted as limiting in nature.

FIG. 61 shows an existing form of the assignee's VOLAR BEARING PLATE™, previously identified as 202. The implant 202 is commonly used in conjunction with a block guide 302 having an inverted shape which is complementary to, and receives, a portion 304 of the implant 202 at which threaded through bores 306 are formed.

The block guide 302 has through bores 308 registrable, one each, with the through bores 306 with the block guide 302 operatively positioned over the implant 202.

A threaded fastener 310 is directed through one of the through bores 308a, that is threaded, and in turn threadably engages an aligned through bore 306a on the implant 202, thereby maintaining the block guide 302 in its operative position. The threaded fastener 310 has a guide passage 312 therethrough which facilitates guided passage of a drilling tool DT, with “DT” intended to encompass all forms of drilling tools, as described above, and others.

The block guide 302 is typically used in combination with a handheld drill guide 314 through which a surgeon can direct an appropriate drilling tool through selected ones of the aligned through bores 306, 308 and into the underlying bone 10.

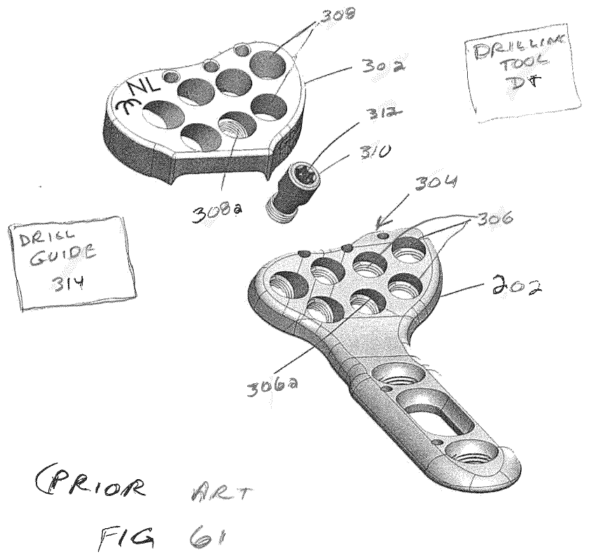

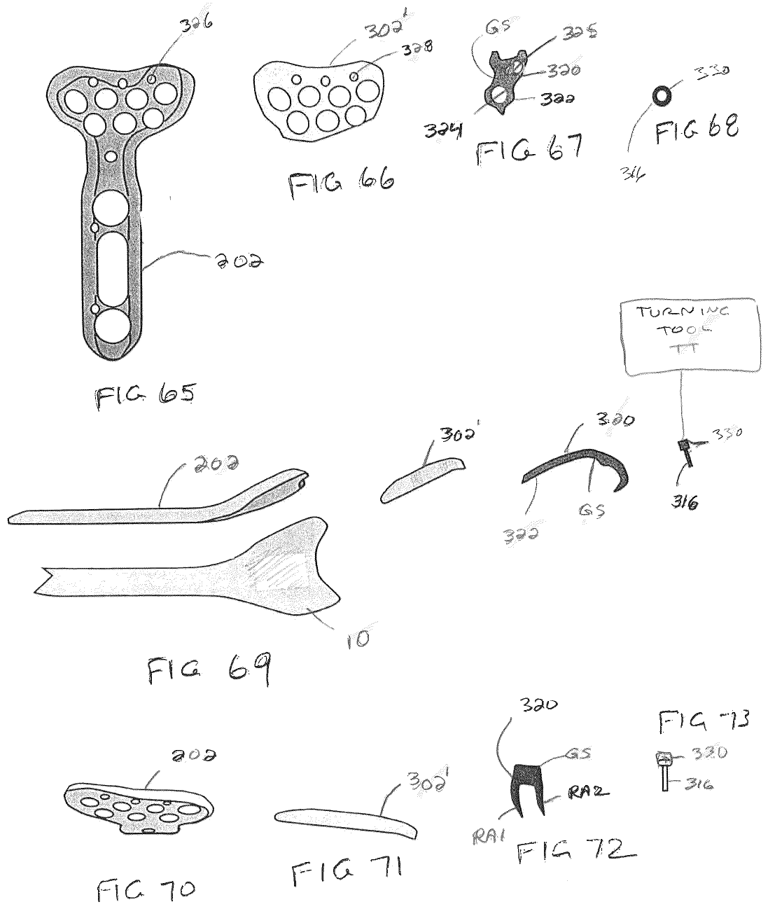

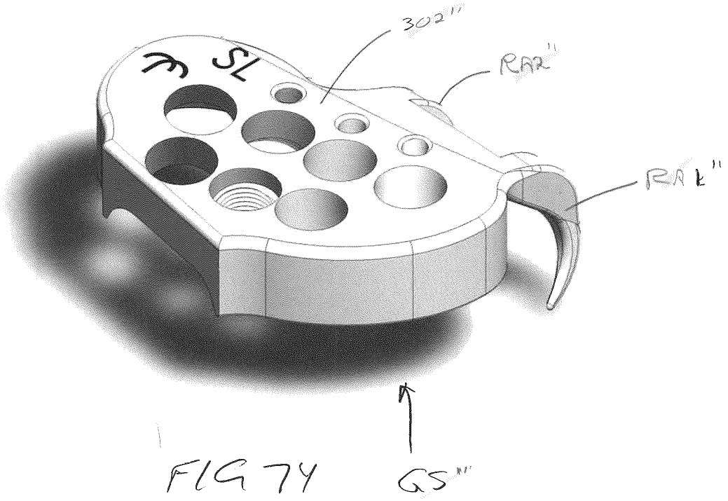

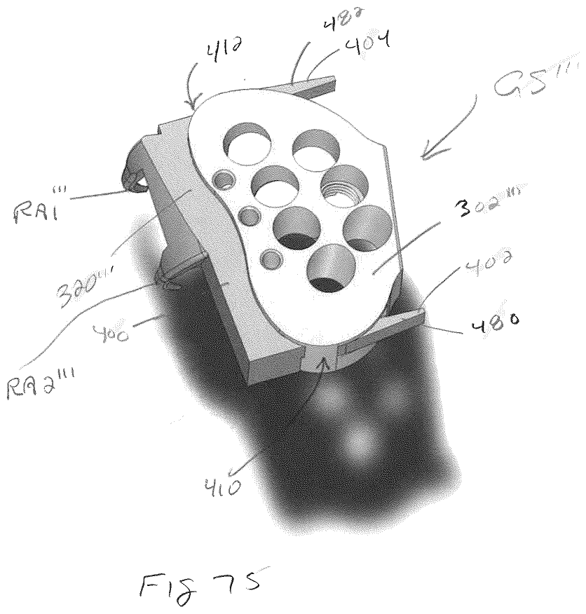

With reference to FIGS. 62-72, a structure that is the same as, or similar to, the block guide 302 is shown as performing, in conjunction with the threaded fastener 310, and a separate threaded coupling bolt/fastener 316, the function of the connector(s) 300.

The block guide 302′ is operatively connected to the implant I/202, as described for the block guide 302, with reference to FIG. 61.

The guide structure GS in the depicted form consists of a body 320 shown with a generally “J” shape from the FIG. 69 perspective. Spaced referencing arms RA1, RA2 are provided. The body 320 has an anchoring portion 322 with an opening 324 therethrough.

The block guide 302′ may be considered to either a part of the guide structure GS, as shown in dotted lines in FIG. 64, or a part separate therefrom.

With the guide structure GS in the operative position of FIGS. 62-64, the opening 324 on the anchoring portion 322 registers with an aligned pair of openings 306, 308. The opening 324 is dimensioned so that with the aforementioned threaded fastener 310 directed into a registered through bore pair 306, 308, the fastener 310 will maintain a desired position of the part of anchoring portion 322 over the block guide 302′.

The coupling bolt 316 is directed through an opening 325 on the guide structure GS aligned with through bores 326, 328, that are in registration and on the implant 202 and block guide 302′, respectively. The coupling bolt 316 is threadably engaged with one or both of the implant 202 and block guide 302′.

With this arrangement, the guide structure GS is consistently maintained in the operative position, as shown in FIGS. 62-64.

The guide structure GS is utilized in the same manner as the forms previously described herein to consistently place the implant 202 in a desired position with respect to the underlying bone 10. As depicted, with the GS fixed to the implant 202, the guide structure GS and implant 202 can be controllably moved as a unit.

With the guide structure GS in the operative position and the implant 202 placed in its desired position, appropriate implant fixation can be carried out. The anchoring portion 322 of the guide structure is configured so as not to obstruct any of the registered through bores 306, 308 used to facilitate the placement of drilling components and/or final fasteners.

Preliminary or final fixation of the implant 202 can be carried out before the guide structure GS is ultimately separated, including the block guide 302′. It is also contemplated that the implant 202 can be fixed after the guide structure GS is removed.

As noted previously, the invention contemplates that any form of releasable connection between the guide structure GS and implant I can be utilized, utilizing the guide structure GS as previously described to facilitate consistent placement of the implant I in a desired seated position on the bone 10.

Separation of the guide structure GS can be simply carried out by removing the coupling bolt 316, which may be facilitated by providing a graspable head 330, or one that readily accommodates a turning tool TT.

The block guide 302′ can be simply separated by removing the threaded fastener 310 and drawing the block guide 302′ away from the implant portion 304.

As noted, the drilling components may be utilized with the block guide 302′ still in its operative position.

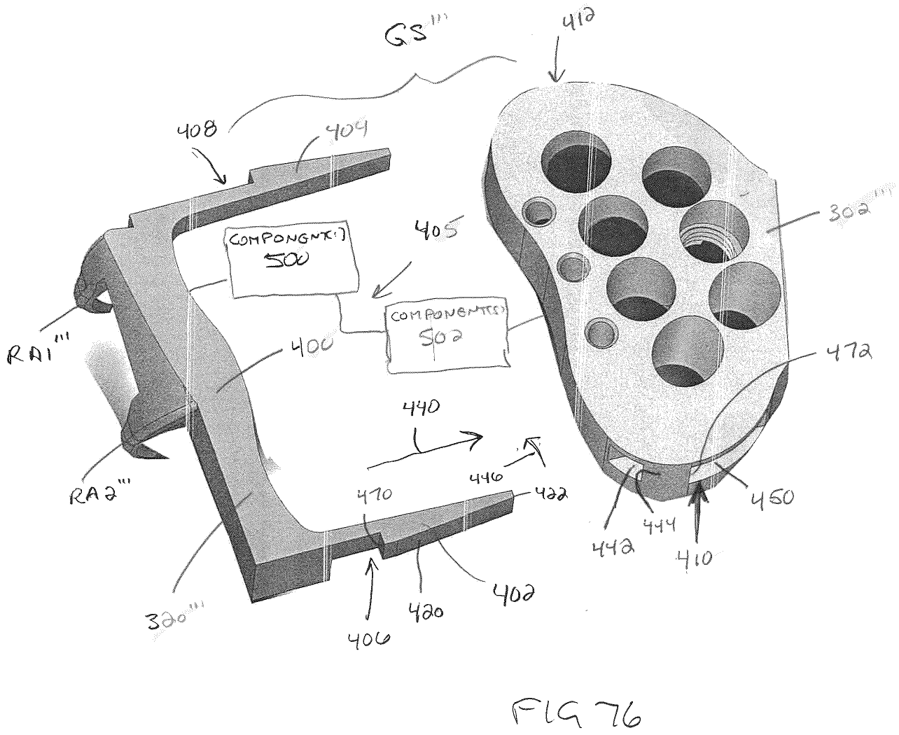

In FIG. 74, a modified form of guide structure, corresponding to the guide structure GS, is shown at GS″. The guide structure GS″ is similar to the guide structure GS, as including the block guide 302′ and body 320, with the primary distinction being that there is a single piece that defines a corresponding block guide 302″ and referencing arms RA1″, RA2″ configured at least substantially the same as the referencing arms RA1, RA2 on the body 320.

In FIGS. 75 and 76 another modified form of guide structure is shown at GS′″. The guide structure GS′″ has a block guide 302′″ and a body 320′″. The body 320′″ has an overall “U” shape with a base 400 of the “U” supporting spaced referencing arms RA1′″, RA2′″ depending therefrom. The legs 402, 404 of the “U” define in conjunction with the base 400 a receptacle 405 for the block guide 302′″.

The legs 402, 404 respectively define connectors 406, 408 that respectively snap fit with connectors 410, 412 on the body 320′″. The snap fit connectors 406, 410 and 408, 412 cooperate in the same fashion.

Exemplary snap fit connector 406 has a cam surface 420 on the leg 402. As a free end 422 of the leg 402 is translated from a starting position, as shown in FIG. 76, spaced from the block guide 302′″, in the direction of the arrow 440 into an entry opening 442 on the snap connector 410, a wall 444 deflects the cantilever mounted leg 402 in the direction of the arrow 446. Continued movement of the leg 402 causes the free end 422 of the leg 402 to move through an exit opening 450, in communication with the entry opening 442, sufficiently that the cam surface 420 moves past the wall 444. A discrete gap 460, trailing the cam surface 420, eventually coincides with the wall 444 at which point restoring forces on the deformed/bent leg 402 cause the leg 402 to spring at least partially back to an undeformed state. As this occurs, oppositely facing surfaces 470, 472, respectively on the leg 402 and wall 444, are placed in confronting relationship so as to block translation of the body 320′″ relative to the block guide 302′″ in a direction opposite to that indicated by the arrow 440 in FIG. 76.

Separation of the base 320′″ from the block guide 302′″, once in the FIG. 75 relationship, is effected by bending the projecting free end regions 480, 482 of the legs 402, 404, respectively, towards each other to release the snap fit connections, whereupon the body 320′″ and block guide 302′″ may be drawn away from each other.

For purposes of stabilization and/or alignment, one or more components 500 may be provided on the body 320′″ to cooperate with one or more connectors 502 on the block guide 302′″. The components may have different cooperating shapes such as, but without limitation, one or more male projections that can be press fit into cooperating and complementary female receptacles as an incident of the body 320′″ and block guide 302′″ being press fit together.

The foregoing disclosure of specific embodiments is intended to be illustrative of the broad concepts comprehended by the invention.

1. A method of fixing an implant with respect to first and second bone parts at a joint with an adjacent bone, the first and second bone parts making up one bone with a length, the implant having a bone engaging surface, the method comprising the steps of:
obtaining a guide structure having a body with at least a first discrete projecting referencing arm,
placing the guide structure in an operative position by causing the at least first discrete projecting referencing arm to penetrate a joint capsule between the first bone part and the adjacent bone and position adjacent a joint surface on the first bone part to abut the joint surface on the first bone part so as to thereby limit shifting of the guide structure relative to the first bone part lengthwise of the one bone and provide a referencing location with respect to the joint surface on the first bone part;
with the guide structure in the operative position, using the guide structure to facilitate alignment of the implant in a desired seated position wherein the bone engaging surface on the implant is in confronting relationship with a surface on the one bone; and
fixing the implant in the desired seated position.

2. The method of fixing an implant according to claim 1 wherein the step of using the guide structure to facilitate alignment of the implant in a desired seated position comprises abutting the implant to the guide structure with the guide structure in the operative position.

3. The method of fixing an implant according to claim 1 wherein the step of using the guide structure to facilitate alignment of the implant in a desired seated position comprises using the guide structure to guidingly direct a drilling tool into the first bone part with the guide structure in the operative position.

4. The method of fixing an implant according to claim 3 wherein the step of using the guide structure to facilitate alignment of the implant in a desired seated position further comprises the step of abutting the implant to the drilling tool with the drilling tool directed into the first bone part.

5. The method of fixing an implant according to claim 3 wherein the step of using the guide structure to facilitate alignment of the implant in a desired seated position comprises separating the drilling tool from the first bone part so that a bore formed by the drilling tool in the first bone part is exposed, and using the bore to one of: a) accept a fastener that can be directed through the implant to fix the implant in the desired seated position; and b) accept a projecting part on the implant.

6. The method of fixing an implant according to claim 1 wherein with the guide structure in the operative position a surface portion on the guide structure bears against a selected region of a surface on the first bone part at a location spaced from a location at which the at least first discrete projecting reference arm abuts the joint surface on the first bone part.

7. The method of fixing an implant according to claim 1 wherein the guide structure comprises a contoured surface configured to bear against a selected region of a surface on the first bone part, the contoured surface defined at least in part by the at least first discrete projecting referencing arm and at least nominally matched in shape to the selected region on the surface of the first bone part.

8. The method of fixing an implant according to claim 7 wherein with the guide structure in the operative position the contoured surface is placed in confronting relationship with the selected region on the surface of the first bone part.

9. The method of fixing an implant according to claim 1 wherein the guide structure has at least a first guide passage, the step of using the guide structure to facilitate alignment of the implant in the desired seated position comprises: a) directing a drilling tool through the first guide passage and into the first bone part with the guide structure in the operative position; and b) with the drilling tool directed into the first bone part extending the drilling tool into an opening in the implant whereby the drilling tool abuts the implant to align the implant along the length of the one bone.

10. The method of fixing an implant according to claim 9 wherein the guide passage has an axis, with the guide structure in the operative position the guide passage axis intersects the first bone part at a predetermined angle with respect to the first bone part, and the step of directing the drilling tool through the first guide passage comprises directing the drilling tool guidingly through the first guide passage along the guide passage axis.

11. The method of fixing an implant according to claim 9 further comprising the step of maintaining the guide structure in the operative position before using the guide structure to facilitate alignment of the implant in the desired seated position.

12. The method of fixing an implant according to claim 11 wherein the step of maintaining the guide structure in the operative position comprises directing a drilling tool through a second guide passage in the guide structure and into the first bone part.

13. The method of fixing an implant according to claim 1 wherein the implant has at least one projecting part, wherein the guide structure body has at least a first guide passage with an axis, wherein with the guide structure in the operative position, the axis of the first guide passage intersects the first bone part at a predetermined location spaced from the referencing location lengthwise of the one bone and at a predetermined angle with respect to the first bone part, wherein the step of using the guide structure to facilitate alignment of the implant comprises: a) maintaining the guide structure in the operative position; and b) with the guide structure maintained in the operative position directing a drilling tool guidingly through the first guide passage to form a first bore in the first bone part, the method further comprising the step of advancing the one projecting part on the implant into the first bore to place the implant in the desired seated position, and wherein the step of fixing the implant comprises fixing the implant to the second bone part.

14. The method of fixing an implant according to claim 13 further comprising the step of repositioning the implant to bring a part of the bone engaging surface on the implant into confronting relationship with a surface on the second bone part and thereby causing the first bone part to be repositioned relative to the second bone part.

15. The method of fixing an implant according to claim 13 wherein the at least first discrete projecting referencing arm has a sharp free end.

16. The method of fixing an implant according to claim 13 wherein the at least first discrete projecting referencing arm tapers towards a free end.

17. The method of fixing an implant according to claim 13 wherein the guide structure comprises a second discrete projecting referencing arm spaced from the first discrete projecting referencing arm and configured to penetrate the joint capsule as the guide structure is placed in the operative position.

18. The method of fixing an implant according to claim 17 wherein the guide structure comprises a contoured surface configured to bear against a selected region of a surface on the first bone part and the contoured surface has a hook-shaped portion to extend over the selected surface region on the first bone part.

19. The method of fixing an implant according to claim 13 wherein the guide structure body has a block portion through which the first guide passage is formed and there is a second guide passage formed through the block portion in spaced relationship with the first guide passage, and further including the step of directing a drilling tool through the second guide passage and into the first bone part to define a second bore in the first bone part.

20. The method of fixing an implant according to claim 12 wherein there is a third guide passage formed through the block portion in spaced relationship to the first and second guide passages and further including the step of directing a drilling tool through the third guide passage and into the first bone part.

21. The method of fixing an implant according to claim 1 wherein the guide structure further comprises an elongate handle projecting in cantilever fashion, the elongate handle having a portion configured to be grasped by a user to facilitate holding and repositioning of the guide structure body.

22. The method of fixing an implant according to claim 21 wherein the portion configured to be grasped on the elongate handle has a gripping length projecting in a line that is angled with respect to the axis of the first guide passage.

23. The method of fixing an implant according to claim 22 wherein the guide structure body has a guide portion through which a first guide passage for a drilling tool is formed, the elongate handle has an “L” shape with first and second legs, the first leg is connected to the block portion and the second leg extends from the first leg and defines the gripping length.

24. The method of fixing an implant according to claim 13 wherein the step of placing the guide structure in the operative position comprises determining an optimal orientation of a line of the first bore relative to the first bone part and selecting the operative position of the guide structure so that the first guide passage axis is substantially coincident with the line of the first bore.

25. The method of fixing an implant according to claim 24 wherein the step of placing the guide structure in the operative position comprises repositioning the guide structure relative to the first bone part with the at least first discrete projection penetrating the joint capsule to thereby change an orientation of the line of the first bore relative to the first bone part to arrive at the optimal orientation of the line of the first bore relative to the first bone part.

26. The method of fixing an implant according to 25 wherein the step of determining an optimal orientation of a line of the first bore comprises making a determination while inspecting the first bone part with x-ray.

27. The method of fixing an implant according to claim 13 wherein the step of advancing the one projecting part on the implant into the first bore comprises guiding the one projecting part along the drilling tool through one of: a) direct interaction of the implant and drilling tool; and b) interaction of the implant and an attachment on the implant.

28. The method of fixing an implant according to claim 13 wherein the drilling tool is separated from the first bone part before advancing the one projecting part on the implant into the first bore.

29. The method of fixing an implant according to claim 14 wherein the step of repositioning the implant comprises directly engaging the implant and exerting a force upon the implant to effect repositioning.

30. The method of fixing an implant according to claim 20 further comprising the step of anchoring the guide structure before advancing the one projecting part on the implant into the first bore.

31. The method of fixing an implant according to claim 30 wherein the step of anchoring the guide structure is performed by directing a drilling tool through one of the first and second guide passages and into the first bone part.

32. The method of fixing an implant according to claim 13 wherein the guide structure is separated from the first bone part before advancing the one projecting part on the implant into the first bore to place the implant in the desired seated position.

33. The method of fixing an implant according to claim 13 wherein the one projecting part on the implant is advanced into the first bore with the guide structure in the operative position.

34. The method of fixing an implant according to claim 13 wherein the implant is in the form of one of: a) a plate with the at least one projecting part having a hook shape; b) a fixed angle plate; c) a plate with the at least one projecting part comprising a fixed projecting part; and d) a body comprising a formed wire.

35. The method of fixing an implant according to claim 13 wherein the implant is guided by the drilling tool towards the desired seated position for the implant.

36. The method of fixing an implant according to claim 35 wherein the guide structure body has a surface that extends only part way around the axis of the first guide passage so that with the drilling tool extending into the first guide passage the guide structure body can be separated from the drilling tool by effecting relative movement of the drilling tool and guide structure body in a direction transverse to the axis of the first guide passage.

37. The method of fixing an implant according to claim 20 further including the step of anchoring the guide structure by directing a drilling tool into one of the guide passages.

38. The method of fixing an implant according to claim 13 wherein the at least first discrete projecting referencing arm has a free end and the axis of the first guide passage is spaced a distance from the free end of the at least first discrete projecting referencing arm.

39. The method of fixing an implant according to claim 38 wherein the guide structure is configured so that the distance between the axis of the first guide passage and free end of the at least first discrete projecting referencing arm can be selectively changed.

40. The method of fixing an implant according to claim 33 wherein the one projecting part is directed through the first guide passage as the one projecting part is advanced into the first bone part.

41. The method of fixing an implant according to claim 13 further comprising the step of providing a kit including at least one of: a) different forms of the implant; b) different forms of the guide structure; c) different forms of the drilling tool; and d) additional components usable to perform the method of claim 6.

42. The method of fixing an implant according to claim 18 wherein the first bone portion defines a rim and the hook-shaped portion extends over the surface on the first bone part at the rim.

43. The method of fixing an implant according to claim 22 wherein the gripping length is oriented so that with the gripping length aligned with a length of the second bone part, the first bone is located and aligned so that with the implant thereafter fixed in the seated position, there is coaptation of the bone engaging surface on the implant along a surface of the second bone part.

44. The method of fixing an implant according to claim 22 wherein the gripping length is oriented so that with the gripping length aligned with a length of the second bone part and the implant thereafter fixed in the desired seated position, a length of the implant aligns with the length of the second bone part.

45. The method of fixing an implant according to claim 1 wherein the guide structure in the operative position is configured to identify a position of an end of the implant with the implant placed in the desired seated position performing the steps of claim 1 using the guide structure in the operative position.

46. The method of fixing an implant according to claim 45 wherein there is a discrete mark on the guide structure facilitating positioning of the end of the implant with the implant placed in the desired seated position.

47. The method of fixing an implant according to claim 1 wherein the guide structure in the operative position is configured to identify an orientation of a length of the implant with the implant placed in the desired seated position performing the steps of claim 1 using the guide structure in the operative position.

48. The method of fixing an implant according to claim 1 wherein the guide structure has an elongate bar that with the guide structure in the operative position projects in a direction substantially aligned with a length of the implant with the implant placed in the desired seated position performing the steps of claim 1 using the guide structure in the operative position.

49. The method of fixing an implant according to claim 13 further comprising the steps of: obtaining an adaptor comprising a body with another guide passage with an axis; and connecting the adaptor to the guide structure so that the adaptor is in an assembled position wherein the axis of the another of the guide passages intersects the first bone part at a location spaced from a location at which the axis of the first guide passage intersects the first bone part.

50. The method of fixing an implant according to claim 49 wherein there are connectors on the adaptor and the guide structure that cooperate to consistently maintain a preselected relationship between the adaptor and guide structure with the adaptor in the assembled position.

51. The method of fixing an implant according to claim 19 further comprising the step of obtaining an adaptor, the adaptor comprising a body with an alignment component and another guide passage with an axis, connecting the adaptor to the guide structure body by directing the alignment component into one of the first and second guide passages and moving the adaptor so that the alignment component advances guidingly in the one of the first and second guide passages until the adaptor achieves an assembled position wherein with the guide structure in the operative position and the adaptor in the assembled position the axis of the another of the guide passages intersects the first bone part at a location spaced from each of: a) a location that the axis of the first guide passage intersects the first bone part, and b) a location that an axis of the second guide passage intersects the first bone part.

52. The method of fixing an implant according to claim 51 wherein the location at which the axis of the another of the guide passages intersects the first bone part is spaced further away from the referencing location than the location at which at least one of the axes of the first and second guide passages intersects the first bone part.

53. The method of fixing an implant according to claim 52 wherein the location at which the axis of the another guide passage intersects the first bone part is spaced further away from the referencing location than the location at which each of the axes of the first and second guide passages intersects the first bone part.

54. The method of fixing an implant according to claim 13 wherein the implant guide structure body has a second guide passage and further comprising the steps of directing a drilling tool guidingly through the second guide passage to form a second bore in the first bone part, removing the drilling tool from the first bore, with the drilling tool remaining in the second bore, directing the drilling tool remaining in the second bore into the one implant opening and thereafter guiding the implant along the drilling tool remaining in the second bore to thereby advance the one projecting part up to the first bore and thereafter into the first bore to place the bone engaging surface on the implant in confronting relationship with the surface on the first bone.

55. The method of fixing an implant according to claim 1 further comprising the step of connecting the body of the guide structure to the implant.

56. The method of fixing an implant according to claim 55 further comprising the step of placing the implant against the one bone after the body of the guide structure is connected to the implant.

57. The method of fixing an implant according to claim 55 wherein the step of connecting the body of the guide structure to the implant comprises the steps of connecting a block guide to the implant and connecting the body of the guide structure to the block guide.

58. The method of fixing an implant according to claim 55 further comprising the step of separating the body of the guide structure from the implant with the implant in the desired seated position.

59. The method of fixing an implant according to claim 57 wherein the step of connecting the body of the guide structure to the implant is performed using at least one separate fastener.

60. The method of fixing an implant according to claim 59 wherein the at least one separate fastener comprises one separate fastener that connects the block guide to the implant.

61. The method of fixing an implant according to claim 59 wherein the at least one separate fastener comprises another one of the at least one of the separate fasteners that connects the body of the guide structure to the block guide.

62. The method of fixing an implant according to claim 57 further comprising the step of separating the block guide from the implant with the implant in the desired seated position.

63. The method of fixing an implant according to claim 59 wherein the at least one separate fastener is a threaded fastener.

64. The method of fixing an implant according to claim 57 wherein the step of connecting the body of the guide structure to the block guide comprises press connecting the body of the guide structure to the block guide.

65. The method of fixing an implant according to claim 64 wherein the step of press connecting the body of the guide structure to the block guide comprises engaging snap fit connectors on the body of the guide structure and the block guide.

66. The guide structure as used in performing the method of claim 1.

67. The guide structure as used to perform the method of claim 54 in combination with a drilling tool configured to be guided by the first guide passage and in the form of one of: a) a rotary drill bit; and b) a pin.

68. The guide structure as used to perform the method of claim 13 wherein the guide structure body has a second guide passage with an axis and with the guide structure in the operative position the axis of the second guide passage intersects the first bone part at a location on the first bone part spaced further away from the referencing location lengthwise of the bone than a location at which the axis of the first guide passage intersects the first bone part.

69. The guide structure as used to perform the method of claim 55.

70. The guide structure including the block guide as used to perform the method of claim 57.

71. The guide structure including the block guide as used to perform the method of claim 65.
